SEQUENCE LISTING (1 sequence)

SEQ ID NO: 1 - DNA, 1655 bp, Homo sapiens
ctgcagcttcactcctgagccagtgagaccacgaacccaccagaaggaagaaactccgaacacatccgaacatcagaaggaacaaactccagacacgccgcctttaagaactgtaacactcaccgcgagggtccgaggcttcattcttgaaggcagtgagaccaagaacccaccaattccggacacagtaccatgaaggaatgaaaatacataacaatgtgatgtatcatgttttatttcctagactagtgacaaatgaaagctaagtgtagcaagggtgcagggacacaggcacatttgtggactaggtgtgagtgtaagctgggttcgatggtcttttggccaacatagtgaacccctgtgtctactaaaaatacaaaaattagccaggcgtggtggtgcaggcctgtagtcccagctacatgggaggctgaggtgggagtatcgcttgaacctgggagacggaagttgcagtgagccgggatcacaccaccgttcaccaatctgagccacagagagactgtctcaaaaaataaaccacaaggaagggagggagggggagggggagggagggaggaaagagaaagagagaaaggaaggaaagagaaagcaggaaggaaggaaagaagaagaaagaagacgaaagaacgaaagaaaaggaaagaagagaggagagaacagaaggggcaggtgcccctgggaaggggagaagatcaagacgcgcctggaaagcggactctgaacctcaagaccctgttcacagccaagcgcgcgaccccgggaggcgtcaactccccaagtgcctccctcaactcatttcccccaagtttcggtgcctgtcctggcgcggacaggacccagaaacaaaccacagcccggggcgcagccgccagggcgaaggttagttccggtcccttcccctcccctccccacttggacgcgcttgcggaggattgcgttgacgagactcttatttattgtcaccaacctgtggtggaatttgcagttgcacattggatctgattcgccccgccccgaatgacgcctgcccggaggcagtgaaagtacagccgcgccgccccaagtcagcctggacacataaatcagcacgcggccggagaaccccgcaatctttgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtgagtccccgccgcggtccctggctggggaagagcgtgcctggcgcctggagagggcagggtagagagggggacacggcgggggtgcgtggcccgggtcgcctgcggccgggcatgtccgggcaagacgcaccagtcgtcggagtcgggggaagagatgggtccccgggttgggcaggagcgacctgggccgccagggaacagagcgcgcgctccacttggtgtaaattcccgaatccagtgggggagggcgacaaggagggaattcccgagtaagctgcgtgaag

(19) United States
(12) Patent Application Publication
(10) Pub. No.: US 2009/0285836 A1
(43) Pub. Date: Nov. 19, 2009

(54) USE OF SALINOSPORAMIDE A TO INHIBIT METASTASIS

(75) Inventors: Stavroula Baritaki, Los Angeles, CA (US); Benjamin Bonavida, Los Angeles, CA (US); Michael Palladino, Olivenhein, CA (US)

Correspondence Address: KNOBBE MARTENS OLSON & BEAR LLP, 2040 MAIN STREET, FOURTEENTH FLOOR, IRVINE, CA, 92614, US

(73) Assignee: NEREUS PHARMACEUTICALS, INC., San Diego, CA (US)

(21) Appl. No.: 12/423,713

(22) Filed: Apr. 14, 2009

Related U.S. Application Data
(60) Provisional application No. 61/057,631, filed on May 30, 2008.
(60) Provisional application No. 61/044,861, filed on Apr. 14, 2008.

(51) Int. Cl. A61K 39/395; A61K 31/40; C12Q 1/68; C12Q 1/02

(52) U.S. Cl. 424/174.1; 514/421; 435/6; 435/29

(57) ABSTRACT

The present invention relates to methods and compositions for treating and evaluating metastatic conditions.

### CROSS-REFERENCE TO RELATED APPLICATIONS

This application claims the benefit of U.S. Provisional Application Ser. No. 61/044,861 filed Apr. 14, 2008, and U.S. Provisional Application Ser. No. 61/057,631 filed May 30, 2008, the disclosures of which are hereby expressly incorporated by reference in their entireties.

### STATEMENT REGARDING FEDERALLY SPONSORED R&D

This invention was made with government support under NIH/NCI research supplements CA107023-02S1 and CA057152-13S1 awarded by the National Institutes of Health and the National Cancer Institute of the United States of America. The government has certain rights in the invention.

### REFERENCE TO SEQUENCE LISTING

The present application is being filed along with a Sequence Listing in electronic format. The Sequence Listing is provided as a file entitled NEREUS182A.TXT, created on Apr. 14, 2009, which is 2.4 KB in size. The information in the electronic format of the Sequence Listing is incorporated herein by reference in its entirety.

### FIELD OF THE INVENTION

The present invention relates to methods and compositions for treating and evaluating metastatic conditions.

### BACKGROUND

Metastatic disease is the primary cause of death for most cancer patients. Metastasis is a process that allows many tumors to expand to different areas of the body, involving either multiple mutations or epigenetic changes. Carcinomas arise from glandular or epithelial cells lining different compartments of the body. Most deaths caused by this class of tumor result from the tumor's metastatic characteristics that allow it to spread to different organs (Pantel K, et al. (2008). “Detection, clinical relevance and specific biological properties of disseminating tumour cells.” Nat Rev Cancer 8: 329-340; Steeg P. S. (2006). “Tumor metastasis: mechanistic insights and clinical challenges.” Nat Med 12: 895-904).

The Epithelial to Mesenchymal Transition (EMT) process is the principal way through which metastasis occurs, beginning with a disruption of intercellular contacts and the enhancement of cell motility, and resulting in the release of cells from the parent epithelial tissue. Epithelial cells lose their association with epithelial cell sheets and acquire many of the attributes of mesenchymal cells including acquisition of increased invasiveness and resistance to apoptosis (Condeelis J, et al. (2006). “Macrophages: obligate partners for tumor cell migration, invasion, and metastasis.” Cell 124: 263-266; Shook D. et al., (2003) “Mechanisms, mechanics and function of epithelial-mesenchymal transitions in early development.” Mech Dev 120: 1351-1383). When active in cancer cells, the EMT program enables these cells to complete the initial steps of invasiveness in the metastatic cascade, specifically, local invasion, intravasation, survival in the circulation and extravasation. Treatment options currently available are rarely able to cure or inhibit metastatic cancer. Accordingly there is a need for therapies to treat metastatic conditions.

### SUMMARY

The present invention relates to methods for treating metastatic conditions and methods for evaluating the metastatic potential of a cancer. Some methods include inhibiting metastasis in a subject having a metastatic condition. Some methods for treating a metastatic condition or inhibiting metastasis include administering to a subject in need thereof an effective amount of a compound of Formula I, or a pharmaceutically acceptable salt or pro-drug ester thereof, wherein the compound of Formula I has the structure:

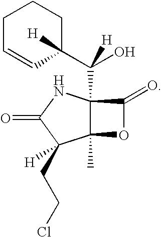

In some methods, the metastatic condition is selected from the group comprising prostate cancer, lung cancer, breast cancer, melanoma, colon cancer, kidney cancer, and pancreatic cancer. In some such methods, the metastatic condition comprises prostate cancer.

In some methods, the compound of Formula I is administered in combination with an effective amount of an additional anticancer agent. In some such methods, the additional anticancer agent is selected from the group comprising vincristine, cis-diamminedichloridoplatinum(II) (CDDP), Tumor necrosis (TNF)-related apoptosis-inducing ligand (TRAIL), and agonist antibodies to DR4 and/or DR5.

In some methods, the subject is a mammal. In more methods, the subject is human.

More embodiments can include methods for evaluating the metastatic potential of a cancer in a subject. Some such methods can include measuring the expression level of at least one marker in a sample from the subject, wherein the at least one marker is selected from RKIP, Snail, NF-κB, E-cadherin, cytokeratin 18, fibronectin, and vimentin.

In some methods, the measuring of the expression level of at least one marker comprises measuring the expression level of a nucleic acid. In more such methods, the measuring of the expression level of at least one marker comprises measuring the expression level of a protein. In even more such methods, the measuring of the expression level of a protein comprises measuring the DNA binding activity of the protein.

Some methods can further include comparing the expression level of said at least one marker in said sample to the expression level of said at least one marker in normal tissue, tissue from a known stage of cancer, or cancerous tissue with a known metastatic potential.

In some methods, the at least one marker comprises RKIP. In some such methods, the at least one marker comprises snail. In more such methods, a decrease in the expression level of said RKIP indicates the metastatic potential of said cancer.

In some methods, the at least one marker comprises at least three markers, at least four markers, at least five markers, or at least six markers.

Some methods can further include administering to a subject in need thereof an effective amount of a compound of Formula I, or a pharmaceutically acceptable salt or pro-drug ester thereof, wherein the compound of Formula I has the structure:

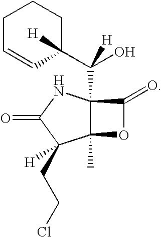

### BRIEF DESCRIPTION OF THE DRAWINGS

FIG. 1 shows a schematic diagram of pathways by which NPI-0052 may modulate tumor metastasis.

FIG. 2A shows graphs of luciferase activity in PC-3 cells, Ramos cells, and DU145 cells, transfected with NFκB-luciferase reporter constructs. Untransfected controls include medium only and DMSO. FIG. 2B shows Western blots of transfected PC-3 and transfected Ramos cells for NF-κB-induced inhibition of DR5 expression.

FIG. 3 shows a graph of percentage apoptosis with increasing concentrations of NPI-0052 in cells treated with cis-diamminedichloridoplatinum(II) (CDDP) or vincristine.

FIG. 4 shows Western blots of PC-3 cells or Ramos cells treated with 2.5 nM NPI-0052 and inhibition of several gene products that regulate apoptosis and resistance.

FIG. 5 shows Western blots of PC-3 and Ramos cells treated with 2.5 nM NPI-0052, and DU145 and LNCaP cells treated with 50 nM NPI-0052 and induction of RKIP.

FIG. 6 shows graphs of percentage apoptosis with increasing concentrations of NPI-0052 in cells treated with 5 ng/ml or 10 ng/ml Tumor necrosis (TNF)-related apoptosis-inducing ligand (TRAIL).

FIG. 7A shows a graph of luciferase activity in cells transfected with a DR5-luciferase reporter construct and treated with 10 μg/ml DHMEQ, or 3 mM, 4 mM, or 5 nM NPI-0052. Controls included untransfected (UNT) and DMSO. FIG. 7B shows a graph of DR5 expression in PC-3 and Ramos cells treated with 1 nM, 2.5 nM, or 5 nM NPI-0052. FIG. 7C shows Western blots and RT-PCR gels of PC-3 or Ramos cells treated with 2.5 nM NPI-0052 and upregulation of DR5 transcription and expression.

FIG. 8 shows a graph of percentage apoptosis in PC-3 cells transfected with CNTR siRNA or RKIP siRNA, and treated with 5 ng/ml TRAIL, 2.5 nM NPI-0052, or 5 ng/ml TRAIL and 2.5 nM NPI-0052. Demonstration of the role of NPI-0052-induced expression of RKIP in sensitization to TRAIL apoptosis.

FIG. 9A shows a RT-PCR gel and Western blot of PC-3 cells transfected with CMV-EV or CMV-RKIP expression constructs, or treated with CDDP. FIG. 9B shows a graph of DR5 surface expression in PC-3 cells transfected with CMV-RKIP expression constructs. FIG. 9C shows a Western blot of PC-3 cells transfected with CMV-RKIP or CMV-EV expression constructs and overexpression of RKIP. FIG. 9D shows an increase of percentage apoptosis in cells transfected with CMV-RKIP but not with CMV-EV expression constructs, and treated with 0 ng/ml, 10 ng/ml, or 15 ng/ml TRAIL.

FIG. 10 shows the sequence of the DR5 promoter (SEQ ID NO:1) (Yoshida T. et al. (2001) “Promoter structure and transcription initiation sites of the human death receptor 5/TRAIL-R2 gene.” FEBS Lett. 507:381-385, incorporated by reference in its entirety)

FIG. 11A shows a graph of luciferase activity in cells transfected with a pYY1-luciferase reporter construct, and treated with DHMEQ or 1 mM, 2 mM, 3 mM, 4 mM, or 5 nM NPI-0052. Both agents inhibited YY1 luciferase activity. Untransfected controls include Medium only, or DMSO. FIG. 11B shows Western blots and RT-PCR gels of PC-3 cells or Ramos cells treated with 2.5 nM NPI-0052 and inhibition of YY1 transcription and expression.

FIG. 12A shows graphs of percentage apoptosis in PC-3 and Ramos cells transfected with YY1 siRNA and treated with 1 ng/ml, 2.5 ng/ml, or 5 ng/ml TRAIL. Also shown are Western blots of PC-3 and Ramos cells transfected with YY1 siRNA. FIG. 12B shows graphs of DR5 expression in PC-3 cells or Ramos cells transfected with YY1 siRNA and upregulation of DR5. Transfection controls include medium only and siRNA negative control.

FIG. 13A shows a graph of luciferase activity in cells transfected with a YY1-lucificerase reporter construct, and CMV-EV or CMV-RKIP expression constructs and CMV-RKIP inhibited YY1 luciferase activity. Controls include transfected with the YY1-lucificerase reporter construct only, and cells treated with 10 μg/ml DHMEQ only. FIG. 13B shows a PT-PCR gel and Western blot of cells transfected with a YY1-lucificerase reporter construct, and CMV-EV or CMV-RKIP expression constructs. CMV-RKIP inhibited both mRNA transcription and protein expression of YY1.

FIG. 14A shows Western blots of LNCaP cells, PC-3 cells, and DU145 cells treated with 0 nM, 2.5 nM, or 50 nM NPI-0052. FIG. 14B shows a Western blot of PC3 cells and DU145 cells transfected with siLuc or siSnail.

FIG. 15A shows a Western blot of DU145 cells and LNCaP cells treated or untreated (−) with NPI-0052. Treatment with NPI-0052-induced the expression of epithelial markers and inhibited mesenchymal markers. FIG. 15B shows a RT-PCR gel of LNCaP cells transfected with f-Snail or f-Snail S6A expression constructs. Treatment with f-Snail S6A inhibited E-cadherin and induced RKIP expression.

FIG. 16A shows a graph of percentage cell viability of DU-145 cells treated with NPI-0052 and resulted in the inhibition of NF-κB lucerase activity. FIG. 16B shows a graph of luciferase activity in DU-145 cells transfected with NF-κB-luciferase reporter constructs and treated with various concentrations of NPI-0052. FIG. 16C shows a Western blot of DU-145 cells treated with 50 nM NPI-0052 for the indicated time points and inhibition of gene products that activate NF-κB.

FIG. 17A shows photomicrographs of DU145 and LNCaP cells treated with NPI-0052. FIG. 17B shows a Western blot derived from DU-145 and LNCaP cells treated with NPI-0052. FIG. 17C shows immunofluorescent micrographs of DU145 and LNCaP cells treated with NPI-0052.

FIG. 18A shows a graph of fold migration of DU-145 cells treated with 50 nM NPI-0052 and/or 100 ng/ml of TNFα. FIG. 18B shows percent invasion for DU-145 cells treated with 50 nM NPI-0052 and/or 100 ng/ml of TNFα. Right panel shows photographs of DU-145 cells treated with 50 nM NPI-0052 and/or 100 ng/ml of TNFα.

FIG. 19A shows a graph of relative RKIP expression in DU-145 cells treated with 50 nM NPI-0052 for indicated times. FIG. 19B shows a Western blot for RKIP expression in DU-145 cells treated with 50 nM NPI-0052 for indicated times. FIG. 19C shows photomicrographs of DU-145 cells transfected with CMV-HA-EV or CMV-HA-RKIP expression constructs. FIG. 19D shows a Western blot of DU-145 cells transfected with CMV-HA-EV or CMV-HA-RKIP expression constructs. FIG. 19E shows immunofluorescent micrographs of DU-145 cells transfected with CMV-HA-EV or CMV-HA-RKIP expression constructs.

FIG. 20A shows a graph of relative Snail expression in DU-145 cells treated with 50 nM NPI-0052 for indicated times. FIG. 20B shows a Western blot of DU-145 cells treated with 50 nM NPI-0052 for indicated times. FIG. 20C shows photomicrographs of DU-145 cells transfected with Snail siRNA or CNTR siRNA. FIG. 20D shows a Western blot of DU-145 cells transfected with Snail siRNA or CNTR siRNA. FIG. 20E shows immunofluorescent micrographs of DU-145 cells transfected with Snail siRNA or CNTR siRNA.

FIG. 21A shows a graph of percentage cell viability in LNCaP cells treated with NPI-0052. FIG. 21B shows a Western blot of LNCaP cells treated with 50 mM NPI-0052, showing an induction of RKIP concomitantly with inhibition of Snail. FIG. 21C shows a Western blot of LNCaP cells transfected with CMV-f-Snail, CMV-f-Snail-6SA, or CMV-EV expression constructs. CMV-f-Snail-6SA induced the EMT phenotype in the non-metastatic LnCAP. FIG. 21D shows a Western blot of DU-145 cells were treated with 5 or 10 μg/ml DHMEQ, mimicking NPI-induced inhibition of NF-κB.

### DETAILED DESCRIPTION

Numerous references are cited herein. The references cited herein, including the U.S. patents cited herein, are each to be considered incorporated by reference in their entirety into this specification.

Treatment of metastasis remains a major problem in cancer and, hence, the urgent need to identify targets for intervention. Tumor cell metastasis is associated with the loss of epithelial features and the acquisition of mesenchymal characteristics and invasive properties by tumor cells, a process known as Epithelial to Mesenchymal Transition (EMT). The activation of the NF-κB pathway has been associated with tumor progression and metastasis. The expression of the Snail transcription factor, through NF-κB activation, is a determinant in the acquisition of EMT through inhibition of metastasis suppressor proteins such as E-cadherin and the regulation of mesenchymal-related gene products.

As demonstrated by the data depicted herein, treatment of the prostate metastatic tumor cell line DU145 with Salinosporamide A resulted in significant inhibition of Snail concomitant with upregulation of RKIP expression and induction of the epithelial gene markers E-cadherin and cytokeratin18, significant inhibition of mesenchymal related gene products such as vimentin and fibronectin and change in cell morphology from a mesenchymal to a more epithelial phenotype. Inhibition of Snail by siRNA or by overexpression of RKIP resulted in similar changes observed above with Salinosporamide A. The direct role of NF-κB-induced inhibition of Snail and reversal of the metastatic phenotype was corroborated by the use of the NF-κB inhibitor, DHMEQ. These findings reveal the role of the proteasome inhibitor, Salinosporamide A, in the regulation and inhibition of the initiation of the metastatic cascade.

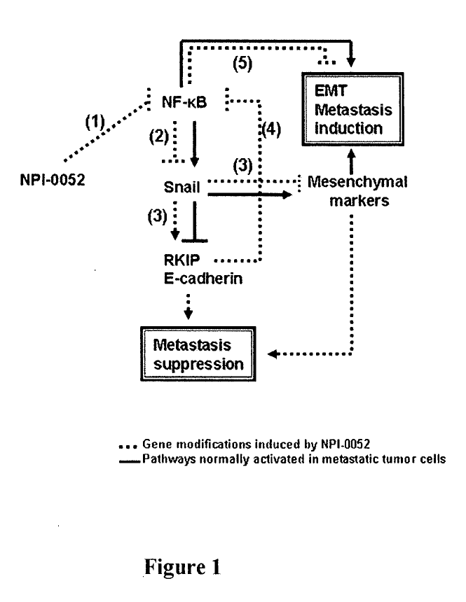

FIG. 1 shows a schematic diagram illustrating the pathways by which the proteasome inhibitor NPI-0052 can regulate and reverse the initiation of tumor metastasis. Proteasome inhibitors mediate their biological effect mainly by inhibiting the NF-κB pathway and consequently the expression of NF-κB-regulated gene products. The Snail transcription factor, an essential initiator of EMT, is under the positive regulation of NF-κB and inhibits the expression of metastasis suppressor genes such as RKIP and E-cadherin, while it induces directly and/or indirectly the expression of mesenchymal markers including vimentin and fibronectin, resulting in the acquisition of a metastatic phenotype by the tumor cells. Based on the data provided herein, in addition to the direct NPI-0052-induced NF-κB inhibition, NF-κB may also be inhibited by NPI-0052-mediated RKIP induction resulting in the modulation of tumor cell metastatic potential. RKIP induction by NPI-0052 may result from down-regulation of its transcriptional repressor Snail via inhibition of its upstream activator NF-κB by NPI-0052 (feedback loop). Snail suppression by NPI-0052 not only induces metastasis suppressor and epithelial gene products, but also represses the expression of Snail-regulated mesenchymal markers, resulting in reversal of the mesenchymal cell phenotype and inhibition of the migratory and invasive properties of the tumor cells.

Described herein are methods for using Salinosporamide A or analogs thereof to reverse tumor resistance to chemotherapy and immunotherapy and inhibit tumor metastasis. One embodiment includes the use of Salinosporamide A or analogs thereof to inhibit epithelial to mesenchymal transition (EMT).

Some embodiments include use of Salinosporamide A in combination with cytotoxic therapeutics in the treatment of resistant and metastatic tumors.

### Salinsporamide A

Salinosporamide A (also known as NPI-0052) has the structure:

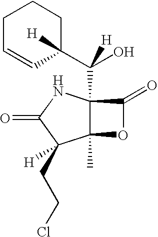

Salinosporamide A and several analogs, as well as methods of making the same are described in U.S. Provisional Patent Applications Nos. 60/480,270, filed Jun. 20, 2003; 60/566,952, filed Apr. 30, 2004; 60/627,461, filed Nov. 12, 2004; 60/633,379, filed Dec. 3, 2004; 60/643,922, filed Jan. 13, 2005; 60/658,884, filed Mar. 4, 2005; 60/676,533, filed Apr. 29, 2005; 60/567,336, filed Apr. 30, 2004; 60/580,838, filed Jun. 18, 2004; 60/591,190, filed Jul. 26, 2004; 60/627,462, filed Nov. 12, 2004; 60/644,132, filed Jan. 13, 2005; 60/659,385, filed Mar. 4, 2005; 60/790,168, filed Apr. 6, 2006; 60/816,968, filed Jun. 27, 2006; 60/836,166, filed Aug. 7, 2006; 60/844,132, filed Sep. 12, 2006; and 60/855,379, filed Jan. 17, 2007; U.S. patent applications Ser. Nos. 10/871,368, filed Jun. 18, 2004 and 11/118,260, now U.S. Pat. No. 7,276,530, filed Apr. 29, 2005; 11/865,704, filed Oct. 1, 2007; 11/412,476, filed Apr. 27, 2006; 11/453,374, filed Jun. 15, 2006; and 11/697,689, filed Apr. 6, 2007; and International Patent Applications Nos, PCT/US2004/019543, filed Jun. 18, 2004; PCT/US2005/044091, filed Dec. 2, 2005, PCT/US2005/01484, filed Apr. 29, 2005, PCT/US2006/016104, filed Apr. 27, 2006; and PCT/US2007/008562, filed Apr. 6, 2007; each of which is hereby incorporated by reference in its entirety.

Salinosporamide A is a potent 20S proteasome inhibitor that is currently in clinical development for the treatment of cancer. The compound and its analogs have various biological activities, for example, the compounds have chemosensitizing activity, anti-microbial, anti-inflammation, radiosensitizing, and anti-cancer activity. Studies show that Salinosporamide A and its analogs have proteasome inhibitory activity, effect NF-κB/IκB signaling pathway, and have anti-anthrax activity (Feling et al., “Salinosporamide A: A Highly Cytotoxic Proteasome Inhibitor from a Novel Microbial Source, a Marine Bacterium of the New Genus Salinospora” Angew. Chem. Int. Ed. 42: 355-357 (2003); Venkat et al., “Structure-Activity Relationship Studies of Salinosporamide A (NPI-0052), a Novel Marine Derived Proteasome Inhibitor” J. Med. Chem. 48: 3684-3687 (2005); and Chauhan et al., “A Novel Proteasome Inhibitor NPI-0052 as an Anticancer Therapy” British Journal of Cancer 95: 961-965 (2006), incorporated by reference in their entireties).

Salinosporamide A can be obtained by fermentation, synthesis, or semi-synthesis. Methods for obtaining Salinosporamide A and its analogs by synthesis are described, for example, in U.S. Patent Publication No. 2007-0249693 titled “Total synthesis of Salinosporamide A and analogs thereof,” filed on Apr. 6, 2007, and U.S. Patent Publication No. 2006-0287520 titled “Synthesis of Salinosporamide A and analogues thereof,” filed on May 16, 2006, which are incorporated by reference in their entireties.

Methods for obtaining Salinosporamide A and its analogs by fermentation are described, for example, in International Patent Application No. PCT/US2006/034930 titled “Biosynthesis of Salinosporamide A and its Analogs” published on Mar. 15, 2007, which is incorporated by reference in its entirety. Examples of techniques include high-yield saline fermentation of the bacteria genus Salinospora, and specific organisms such as CNB476 (ATCC patent deposition number PTA-5275), and NPS21184, a strain derived from CNB476.

In some embodiments, analogs of Salinosporamide A can be obtained by modifying fermentation conditions to yield analogs in the fermentation extracts. More analogs can be generated through directed biosynthesis. Directed biosynthesis is the modification of a natural product by adding biosynthetic precursor analogs to the fermentation of producing microorganisms (Lam, et al., J Antibiot (Tokyo) 44:934 (1991), Lam, et al., J Antibiot (Tokyo) 54:1 (2001); which are hereby incorporated by reference in their entireties). More analogs of Salinosporamide A can also be obtained by biotransformation. Biotransformation reactions are chemical reactions catalyzed by enzymes or whole cells containing these enzymes (Zaks, A., Curr Opin Chem Biol 5:130 (2001), incorporated by reference in its entirety). Microbial natural products are ideal substrates for biotransformation reactions as they are synthesized by a series of enzymatic reactions inside microbial cells. Riva, S., Curr Opin Chem Biol 5:106 (2001).

Salinosporamide A has various biological activities, for example, chemosensitizing activity, anti-microbial, anti-inflammation, radiosensitizing, anti-cancer activity, and proteasome inhibitory activity. The proteasome inhibitory activity may, in whole or in part, contribute to the ability of the compounds to act as anti-cancer, anti-inflammatory, and anti-microbial agents.

The proteasome is a multi-subunit protease that degrades intracellular proteins through its chymotrypsin-like, trypsin-like and peptidylglutamyl-peptide hydrolyzing (PGPH; and also known as the caspase-like activity) activities. The 26S proteasome contains a proteolytic core called the 20S proteasome and one or two 19S regulatory subunits. The 20S proteasome is responsible for the proteolytic activity against many substrates including damaged proteins, the transcription factor NF-κB and its inhibitor IκB, members of the BCL-2 family, signaling molecules, tumor suppressors, including p53, and cell cycle regulators, including p21 and p27.

In one study, Salinosporamide A displayed potent in vitro cytotoxicity against HCT-116 human colon carcinoma with an IC₅₀ value of 11 ng/mL (Feling et al., “Salinosporamide A: A Highly Cytotoxic Proteasome Inhibitor from a Novel Microbial Source, a Marine Bacterium of the New Genus Salinospora” Angew. Chem. Int. Ed. 42: 355-357 (2003). incorporated by reference in its entirety). Salinosporamide A also displayed potent and highly selective activity in the National Cancer Institute's 60-cell-line panel with a mean GI₅₀ value (the concentration required to achieve 50% growth inhibition) of less than 10 nm and a greater than 4 log LC₅₀ differential between resistant and susceptible cell lines. The greatest potency was observed against NCI-H226 non-small cell lung cancer, SF-539 CNS cancer, SK-MEL-28 melanoma, and MDA-MB-435 breast cancer (all with LC₅₀ values less than 10 nm). Salinosporamide A was tested for its effects on proteasome function. When tested against purified 20S proteasome, Salinosporamide A inhibited proteasomal chymotrypsin-like proteolytic activity with an IC₅₀ value of 1.3 nm. This compound is approximately 35 times more potent than Omuralide (IC₅₀=49 nm), a beta-lactone derived from the naturally occurring lactacystin with structural similarity to Salinosporamide A.

Another study demonstrates that tumor cells can be more sensitive to proteasome inhibitors than normal cells. Moreover, proteasome inhibition increases the sensitivity of cancer cells to anticancer agents. In this study, a panel of cell lines including human colorectal (HT-29 and LoVo), prostate (PC3), breast (MDA-MB-231), lung (NCI-H292), ovarian (OVCAR3), acute T-cell leukemia (Jurkat), murine melanoma (B 16-Fl O) and normal human fibroblasts (CCD-27sk) was treated with Salinosporamide A for 48 hours to assess cytotoxic activity. Tumor cells were sensitive to Salinosporamide A treatment, with HT-29, LoVo, PC3, MDA-MB-231, NCI-H292, OVCAR3, Jurkat, and B16-F10 cells having EC₅₀ values of 47, 69, 78, 67, 97, 69, 10, and 33 mM, respectively. In contrast, normal fibroblasts, CCD-27sk cells had an EC₅₀ value of 196 nM. It was also observed that treatment of Jurkat cells with Salinosporamide A at the approximate EC₅₀ resulted in Caspase-3 activation and cleavage of PARP confirming the induction of apoptosis.

Additional studies have characterized the effects of Salinosporamide A on the NF-κB signaling pathway. The 20S proteasome regulates the activity of the transcription factor NF-κB. NF-κB promotes cell survival by regulating genes encoding anti-apoptotic proteins. In its inactive form, NF-κB complexes with its inhibitor IκB, but upon stimulation, IκB is degraded by the proteasome, leading to NF-κB activation. NF-κB is constitutively active in many tumors. Interference with NF-κB activity by proteasome inhibition results in enhanced chemosensitivity and increased apoptosis in cancer cells (Cusack et al., “Enhanced chemosensitivity to CPT-11 with proteasome inhibitor PS-341: implications for systemic nuclear factor kapp B inhibition.” Cancer Res. (2001) 61: 3535-3540).

### Pharmaceutical Compositions

In some embodiments, the compounds disclosed herein are used in pharmaceutical compositions. The compounds preferably can be produced by the methods disclosed herein. The compounds can be used, for example, in pharmaceutical compositions comprising a pharmaceutically acceptable carrier prepared for storage and subsequent administration. Also, embodiments relate to a pharmaceutically effective amount of the products and compounds disclosed above in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), which is incorporated herein by reference in its entirety. Preservatives, stabilizers, dyes and even flavoring agents can be provided in the pharmaceutical composition. For example, sodium benzoate, ascorbic acid and esters of p-hydroxybenzoic acid can be added as preservatives. In addition, antioxidants and suspending agents can be used.

The term “pro-drug ester,” especially when referring to a pro-drug ester of the compositions disclosed herein, refers to a chemical derivative of the compound that is rapidly transformed in vivo to yield the compound, for example, by hydrolysis in blood or inside tissues. The term “pro-drug ester” refers to derivatives of the compounds disclosed herein formed by the addition of any of several ester- or thioester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other prodrugs can be prepared by preparing a corresponding thioester of the compound, for example, by reacting with an appropriate thiol, such as thiophenol, Cysteine or derivatives thereof, or propanethiol, for example. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design Theory and Application”, edited by E. B. Roche, Pergamon Press: New York, 14-21 (1987) (providing examples of esters useful as prodrugs for compounds containing carboxyl groups). Each of the above-mentioned references is hereby incorporated by reference in its entirety.

The term “pharmaceutically acceptable salt,” as used herein, and particularly when referring to a pharmaceutically acceptable salt of the compositions disclosed herein, refers to any pharmaceutically acceptable salts of a compound, and preferably refers to an acid addition salt of a compound. Preferred examples of pharmaceutically acceptable salt are the alkali metal salts (sodium or potassium), the alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane. With respect to compounds synthesized by the method of this embodiment that are basic amines, the preferred examples of pharmaceutically acceptable salts are acid addition salts of pharmaceutically acceptable inorganic or organic acids, for example, hydrohalic, sulfuric, phosphoric acid or aliphatic or aromatic carboxylic or sulfonic acid, for example acetic, succinic, lactic, malic, tartaric, citric, ascorbic, nicotinic, methanesulfonic, p-toluensulfonic or naphthalenesulfonic acid.

Preferred pharmaceutical compositions disclosed herein include pharmaceutically acceptable salts and pro-drug esters of the compound of Formula I obtained and purified by the methods disclosed herein. Accordingly, if the manufacture of pharmaceutical formulations involves intimate mixing of the pharmaceutical excipients and the active ingredient in its salt form, then it is preferred to use pharmaceutical excipients which are non-basic, that is, either acidic or neutral excipients.

It will be also appreciated that the phrase “compounds and compositions comprising the compound,” or any like phrase, is meant to encompass compounds in any suitable form for pharmaceutical delivery, as discussed in further detail herein. For example, in certain embodiments, the compounds or compositions comprising the same may include a pharmaceutically acceptable salt of the compound.

The compositions can be formulated and used as tablets, capsules, or elixirs for oral administration; suppositories for rectal administration; sterile solutions, suspensions for injectable administration; patches for transdermal administration, and sub-dermal deposits and the like. Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (for example, liposomes), can be utilized.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Dragee cores are provided with suitable coatings. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses. For this purpose, concentrated sugar solutions can be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses. Such formulations can be made using methods known in the art (see, for example, U.S. Pat. Nos. 5,733,888 (injectable compositions); 5,726,181 (poorly water soluble compounds); 5,707,641 (therapeutically active proteins or peptides); 5,667,809 (lipophilic agents); 5,576,012 (solubilizing polymeric agents); 5,707,615 (anti-viral formulations); 5,683,676 (particulate medicaments); 5,654,286 (topical formulations); 5,688,529 (oral suspensions); 5,445,829 (extended release formulations); 5,653,987 (liquid formulations); 5,641,515 (controlled release formulations) and 5,601,845 (spheroid formulations); all of which are incorporated herein by reference in their entireties.

Further disclosed herein are various pharmaceutical compositions well known in the pharmaceutical art for uses that include topical, intraocular, intranasal, and intraauricular delivery. Pharmaceutical formulations include aqueous ophthalmic solutions of the active compounds in water-soluble form, such as eyedrops, or in gellan gum (Shedden et al., Clin. Ther., 23(3):440-50 (2001)) or hydrogels (Mayer et al., Opthalmologica, 210(2):101-3 (1996)); ophthalmic ointments; ophthalmic suspensions, such as microparticulates, drug-containing small polymeric particles that are suspended in a liquid carrier medium (Joshi, A. 1994 J Ocul Pharmacol 10:29-45), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions may also include drops and sprays often prepared to simulate in many respects nasal secretions to ensure maintenance of normal ciliary action. As disclosed in Remington's Pharmaceutical Sciences (Mack Publishing, 18^{th} Edition), which is incorporated herein by reference in its entirety, and well-known to those skilled in the art, suitable formulations are most often and preferably isotonic, slightly buffered to maintain a pH of 5.5 to 6.5, and most often and preferably include anti-microbial preservatives and appropriate drug stabilizers. Pharmaceutical formulations for intraauricular delivery include suspensions and ointments for topical application in the ear. Common solvents for such aural formulations include glycerin and water.

When used as an anti-cancer compound, for example, the compositions described herein can be administered by either oral or non-oral pathways. When administered orally, it can be administered in capsule, tablet, granule, spray, syrup, or other such form. When administered non-orally, it can be administered as an aqueous suspension, an oily preparation or the like or as a drip, suppository, salve, ointment or the like, when administered via injection, subcutaneously, intraperitoneally, intravenously, intramuscularly, or the like.

In one embodiment, the anti-cancer agent can be mixed with additional substances to enhance their effectiveness.

### Methods of Administration

In some embodiments, the disclosed chemical compounds and the disclosed pharmaceutical compositions are administered by a particular method as an anti-cancer agent. Such methods include, among others, (a) administration though oral pathways, which administration includes administration in capsule, tablet, granule, spray, syrup, or other such forms; (b) administration through non-oral pathways, which administration includes administration as an aqueous suspension, an oily preparation or the like or as a drip, suppository, salve, ointment or the like; administration via injection, subcutaneously, intraperitoneally, intravenously, intramuscularly, intradermally, or the like; as well as (c) administration topically, (d) administration rectally, or (e) administration vaginally, as deemed appropriate by those of skill in the art for bringing the compound of the present embodiment into contact with living tissue; and (f) administration via controlled released formulations, depot formulations, and infusion pump delivery. As further examples of such modes of administration and as further disclosure of modes of administration, disclosed herein are various methods for administration of the disclosed chemical compounds and pharmaceutical compositions including modes of administration through intraocular, intranasal, and intraauricular pathways.

The pharmaceutically effective amount of the compositions that include the described compounds required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

In practicing the methods of the embodiment, the products or compositions can be used alone or in combination with one another, or in combination with other therapeutic or diagnostic agents. These products can be utilized in vivo ordinarily in a mammal, preferably in a human, or in vitro. In employing them in vivo the products or compositions can be administered to the mammal in a variety of ways, including parenterally, intravenously, subcutaneously, intramuscularly, colonically, rectally, vaginally, nasally or intraperitoneally, employing a variety of dosage forms. Such methods may also be applied to testing chemical activity in vivo.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired affects and the therapeutic indication. Typically, dosages can be between about 10 μg/kg and 100 mg/kg body weight, preferably between about 100 μg/kg and 10 mg/kg body weight. Alternatively dosages can be based and calculated upon the surface area of the patient, as understood by those of skill in the art. Administration is preferably oral on a daily or twice daily basis.

The exact formulation, route of administration and dosage can be chosen by the individual physician in view of the patient's condition. See for example, Fingl et al., in The Pharmacological Basis of Therapeutics, 1975, which is incorporated herein by reference in its entirety. It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above can be used in veterinary medicine.

Depending on the specific conditions being treated, such agents can be formulated and administered systemically or locally. A variety of techniques for formulation and administration can be found in Remington's Pharmaceutical Sciences, 18th Ed., Mack Publishing Co., Easton, Pa. (1990), which is incorporated herein by reference in its entirety. Suitable administration routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the embodiment can be formulated in aqueous solutions, preferably in physiologically compatible buffers such as Hanks' solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the embodiment into dosages suitable for systemic administration is within the scope of the embodiment. With proper choice of carrier and suitable manufacturing practice, the compositions disclosed herein, in particular, those formulated as solutions, can be administered parenterally, such as by intravenous injection. The compounds can be formulated readily using pharmaceutically acceptable carriers well known in the art into dosages suitable for oral administration. Such carriers enable the compounds of the embodiment to be formulated as tablets, pills, capsules, liquids, gels, syrups, slurries, suspensions and the like, for oral ingestion by a patient to be treated.

Agents intended to be administered intracellularly can be administered using techniques well known to those of ordinary skill in the art. For example, such agents can be encapsulated into liposomes, then administered as described above. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. Additionally, due to their hydrophobicity, small organic molecules can be directly administered intracellularly.

Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration can be in the form of tablets, dragees, capsules, or solutions. The pharmaceutical compositions can be manufactured in a manner that is itself known, for example, by means of conventional mixing, dissolving, granulating, dragee-making, levitating, emulsifying, encapsulating, entrapping, or lyophilizing processes.

Compounds disclosed herein can be evaluated for efficacy and toxicity using known methods. For example, the toxicology of a particular compound, or of a subset of the compounds, sharing certain chemical moieties, can be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, can be determined using known methods. The efficacy of a particular compound can be established using several art recognized methods, such as in vitro methods, animal models, or human clinical trials. Art-recognized in vitro models exist for nearly every class of condition, including the conditions abated by the compounds disclosed herein, including cancer, cardiovascular disease, and various immune dysfunction, and infectious diseases. Similarly, acceptable animal models can be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

When used as an anti-cancer agent, the compounds disclosed herein can be administered by either oral or a non-oral pathways. When administered orally, it can be administered in capsule, tablet, granule, spray, syrup, or other such form. When administered non-orally, it can be administered as an aqueous suspension, an oily preparation or the like or as a drip, suppository, salve, ointment or the like, when administered via injection, subcutaneously, intraperitoneally, intravenously, intramuscularly, intradermally, or the like. Controlled release formulations, depot formulations, and infusion pump delivery are similarly contemplated.

The compositions disclosed herein in pharmaceutical compositions may also comprise a pharmaceutically acceptable carrier. Such compositions can be prepared for storage and for subsequent administration. Acceptable carriers or diluents for therapeutic use are well known in the pharmaceutical art, and are described, for example, in Remington's Pharmaceutical Sciences, Mack Publishing Co. (A. R. Gennaro edit. 1985). For example, such compositions can be formulated and used as tablets, capsules or solutions for oral administration; suppositories for rectal or vaginal administration; sterile solutions or suspensions for injectable administration. Injectables can be prepared in conventional forms, either as liquid solutions or suspensions, solid forms suitable for solution or suspension in liquid prior to injection, or as emulsions. Suitable excipients include, but are not limited to, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. If desired, absorption enhancing preparations (for example, liposomes), can be utilized.

The pharmaceutically effective amount of the composition required as a dose will depend on the route of administration, the type of animal being treated, and the physical characteristics of the specific animal under consideration. The dose can be tailored to achieve a desired effect, but will depend on such factors as weight, diet, concurrent medication and other factors which those skilled in the medical arts will recognize.

The products or compositions of the embodiment, as described above, can be used alone or in combination with one another, or in combination with other therapeutic or diagnostic agents. These products can be utilized in vivo or in vitro. The useful dosages and the most useful modes of administration will vary depending upon the age, weight and animal treated, the particular compounds employed, and the specific use for which these composition or compositions are employed. The magnitude of a dose in the management or treatment for a particular disorder will vary with the severity of the condition to be treated and to the route of administration, and depending on the disease conditions and their severity, the compositions can be formulated and administered either systemically or locally. A variety of techniques for formulation and administration can be found in Remington's Pharmaceutical Sciences, 18th ed., Mack Publishing Co., Easton, Pa. (1990).

To formulate the anti-cancer agents described herein, known surface active agents, excipients, smoothing agents, suspension agents and pharmaceutically acceptable film-forming substances and coating assistants, and the like can be used. Preferably alcohols, esters, sulfated aliphatic alcohols, and the like can be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like can be used as excipients; magnesium stearate, talc, hardened oil and the like can be used as smoothing agents; coconut oil, olive oil, sesame oil, peanut oil, soya can be used as suspension agents or lubricants; cellulose acetate phthalate as a derivative of a carbohydrate such as cellulose or sugar, or methylacetate-methacrylate copolymer as a derivative of polyvinyl can be used as suspension agents; and plasticizers such as ester phthalates and the like can be used as suspension agents. In addition to the foregoing preferred ingredients, sweeteners, fragrances, colorants, preservatives and the like can be added to the administered formulation of the compound produced by the method of the embodiment, particularly when the compound is to be administered orally.

The compounds and compositions can be orally or non-orally administered to a human patient in the amount of about 0.001 mg/kg/day to about 10,000 mg/kg/day of the active ingredient, and more preferably about 0.1 mg/kg/day to about 100 mg/kg/day of the active ingredient at, preferably, one time per day or, less preferably, over two to about ten times per day. Alternatively and also preferably, the compound produced by the method of the embodiment may preferably be administered in the stated amounts continuously by, for example, an intravenous drip. Thus, for the example of a patient weighing 70 kilograms, the preferred daily dose of the active ingredient would be about 0.07 mg/day to about 700 g/day, and more preferable, 7 mg/day to about 7 g/day. Nonetheless, as will be understood by those of skill in the art, in certain situations it can be necessary to administer the anti-cancer compound of the embodiment in amounts that excess, or even far exceed, the above-stated, preferred dosage range to effectively and aggressively treat particularly advanced cancers or infections.

In the case of using the compounds described herein as a biochemical test reagent, the compound inhibits the progression of the disease when it is dissolved in an organic solvent or hydrous organic solvent and it is directly applied to any of various cultured cell systems. Usable organic solvents include, for example, methanol, methylsulfoxide, and the like. The formulation can, for example, be a powder, granular or other solid inhibitor, or a liquid inhibitor prepared using an organic solvent or a hydrous organic solvent. While a preferred concentration of the compound produced by the method of the embodiment for use as an anticancer compound is generally in the range of about 1 to about 100 μg/ml, the most appropriate use amount varies depending on the type of cultured cell system and the purpose of use, as will be appreciated by persons of ordinary skill in the art. Also, in certain applications it can be necessary or preferred to persons of ordinary skill in the art to use an amount outside the foregoing range.

In one embodiment, the method of using a compound as an anti-cancer agent involves administering an effective amount of Salinosporamide A. In a preferred embodiment, the method involves administering Salinosporamide A to a patient in need of an anti-cancer agent, until the need is effectively reduced or more preferably removed.

As will be understood by one of skill in the art, “need” is not an absolute term and merely implies that the patient can benefit from the treatment of the anti-cancer agent in use. By “patient” what is meant is an organism that can benefit by the use of an anti-cancer agent. For example, any organism with cancer, such as, a metastatic cancer condition, e.g. prostate cancer, breast cancer, lung cancer, kidney cancer, melanoma, etc. The terms “patient” and “subject” may, in some embodiments, be interchangeable. A subject can be mammalian, for example, human, and non-human.

In one embodiment, the patient's health may not require that an anti-cancer agent be administered, however, the patient may still obtain some benefit by the reduction of the level of cancer cells present in the patient, and thus be in need. In one embodiment, the anti-cancer agent is effective against one type of cancer, but not against other types; thus, allowing a high degree of selectivity in the treatment of the patient. In still further embodiments, the anti-cancer agent is effective against a broad spectrum of cancers or all cancers. Examples of cancers, against which the compounds can be effective include CLL, MCL, pancreatic cancer, a colorectal carcinoma, a prostate carcinoma, a breast adenocarcinoma, a non-small cell lung carcinoma, an ovarian carcinoma, multiple myelomas, a melanoma, and the like. In preferred embodiments, the cancer can be metastatic form of cancer.

“Therapeutically effective amount,” “pharmaceutically effective amount,” or similar term, means that amount of drug or pharmaceutical agent that will result in a biological or medical response of a cell, tissue, system, animal, or human that is being sought. In a preferred embodiment, the medical response is one sought by a researcher, veterinarian, medical doctor, or other clinician.

“Anti-cancer agent” refers to a compound or composition including the compounds described herein that reduces the likelihood of survival of a cancer cell. In one embodiment, the likelihood of survival is determined as a function of an individual cancer cell; thus, the anti-cancer agent will increase the chance that an individual cancer cell will die. In one embodiment, the likelihood of survival is determined as a function of a population of cancer cells; thus, the anti-cancer agent will increase the chances that there will be a decrease in the population of cancer cells. In one embodiment, anti-cancer agent means chemotherapeutic agent or other similar term.

A “chemotherapeutic agent” is a chemical compound useful in the treatment of a neoplastic disease, such as cancer. Examples of chemotherapeutic agents include alkylating agents, such as a nitrogen mustard, an ethyleneimine and a methylmelamine, an alkyl sulfonate, a nitrosourea, and a triazene, folic acid antagonists, anti-metabolites of nucleic acid metabolism, antibiotics, pyrimidine analogs, 5-fluorouracil, cisplatin, purine nucleosides, amines, amino acids, triazol nucleosides, corticosteroids, a natural product such as a vinca alkaloid, an epipodophyllotoxin, an antibiotic, an enzyme, a taxane, and a biological response modifier or antibodies to biological response modifiers or other agents; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gouadotropin-releasing hormone analog. Specific examples include Adriamycin, Doxorubicin, 5-Fluorouracil, Cytosine arabinoside (“Ara-C”), Cyclophosphamide, Thiotepa, Busulfan, Cytoxin, Taxol, Toxotere, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins, Melphalan, and other related nitrogen mustards. Also included in this definition are hormonal agents that act to regulate or inhibit hormone action on tumors, such as tamoxifen and onapristone.

Additional examples of such chemotherapeutics include alkaloids, alkylating agents, antibiotics, antimetabolites, enzymes, hormones, platinum compounds, immunotherapeutics (antibodies, T-cells, epitopes), BRMs, and the like. Examples include, Vincristine, Vinblastine, Vindesine, Paclitaxel (Taxol), Docetaxel, topoisomerase inhibibitors epipodophyllotoxins (Etoposide VP-16), Camptothecin, nitrogen mustards (cyclophosphamide Cytoxan), Nitrosoureas, Carmustine, lomustine, dacarbazine, hydroxymethylmelamine, thiotepa and mitocycin C, Dactinomycin (Actinomycin D), anthracycline antibiotics (Daunorubicin, Daunomycin, Cerubidine), Doxorubicin (Adriamycin), Idarubicin (Idamycin), Anthracenediones (Mitoxantrone), Bleomycin (Blenoxane), Plicamycin (Mithramycin, Antifolates (Methotrexate (Folex, Mexate)), purine antimetabolites (6-mercaptopurine (6-MP, Purinethol) and 6-thioguanine (6-TG). The two major anticancer drugs in this category are 6-mercaptopurine and 6-thioguanine, Chlorodeoxyadenosine and Pentostatin, Pentostatin (2′-deoxycoformycin), pyrimidine antagonists, Avastin, Leucovorin, Oxaliplatin, fluoropyrimidines (5-fluorouracil(Adrucil), 5-fluorodeoxyuridine (FdUrd) (Floxuridine)), Cytosine Arabinoside (Cytosar, ara-C), Fludarabine, L-ASPARAGINASE, Hydroxyurea, glucocorticoids, antiestrogens, tamoxifen, nonsteroidal antiandrogens, flutamide, aromatase inhibitors Anastrozole(Arimidex), Cisplatin, 6-Mercaptopurine and Thioguanine, Methotrexate, Cytoxan, Cytarabine, L-Asparaginase, Steroids: Prednisone and Dexamethasone, bevacizumab, and gemcitabine. Also, proteasome inhibitors such as bortezomib, carfilzomib (PR-171), MG132, lactacystin and disulfuram, can be used in combination with the instant compounds, for example. Examples of biologics can include agents such as TRAIL, antibodies to TRAIL and agonistic antibodies TRAIL death receptors DR4 and/or DR5 (Locklin R. M. et al. (2007) “Agonists of TRAIL death receptors induce myeloma cell apoptosis that is not prevented by cells of the bone marrow environment.”Leukemia 21:805-812, incorporated by reference in its entirety), integrins such as alpha-V-beta-3 (αVβ3) and/or other cytokine/growth factors that are involved in angiogenesis, VEGF, EGF, FGF and PDGF and antibodies to these cytokines/growth factors such as Erbitux. In some aspects, the compounds can be conjugated to or delivered with an antibody. Antibodies may be polyclonal or monoclonal.

The anti-cancer agent may act directly upon a cancer cell to kill the cell, induce death of the cell, to prevent division of the cell, and the like. Alternatively, the anti-cancer agent may indirectly act upon the cancer cell by limiting nutrient or blood supply to the cell, for example. Such anti-cancer agents are capable of destroying or suppressing the growth or reproduction of cancer cells, such as a CLL, MCL, colorectal carcinoma, a prostate carcinoma, a breast adenocarcinoma, a non-small cell lung carcinoma, an ovarian carcinoma, multiple myelomas, a melanoma, and the like. In preferred embodiments, the cancer cells can be a metastatic form of cancer.

In one embodiment, a described compound, preferably Salinosporamide A, is considered an effective anti-cancer agent if the compound can influence 10% of the cancer cells, for example. In a more preferred embodiment, the compound is effective if it can influence 10 to 50% of the cancer cells. In an even more preferred embodiment, the compound is effective if it can influence 50-80% of the cancer cells. In an even more preferred embodiment, the compound is effective if it can influence 80-95% of the cancer cells. In an even more preferred embodiment, the compound is effective if it can influence 95-99% of the cancer cells. “Influence” is defined by the mechanism of action for each compound. For example, if a compound prevents the division of cancer cells, then influence is a measure of prevention of cancer cell division. Not all mechanisms of action need be at the same percentage of effectiveness. In an alternative embodiment, a low percentage effectiveness can be desirable if the lower degree of effectiveness is offset by other factors, such as the specificity of the compound, for example. Thus a compound that is only 10% effective, for example, but displays little in the way of harmful side-effects to the host, or non-harmful microbes or cells, can still be considered effective.

By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

### Metastasis—Epithelial to Mesenchymal Transition

The Epithelial to Mesenchymal Transition (EMT) process is the principal way through which metastasis occurs, beginning with a disruption of intercellular contacts and the enhancement of cell motility, and resulting in the release of cells from the parent epithelial tissue. Epithelial cells lose their association with epithelial cell sheets and acquire many of the attributes of mesenchymal cells including acquisition of increased invasiveness and resistance to apoptosis (Condeelis J, Pollard J W. (2006). “Macrophages: obligate partners for tumor cell migration, invasion, and metastasis.” Cell 124: 263-266; Shook D, Keller R. (2003) “Mechanisms, mechanics and function of epithelial-mesenchymal transitions in early development.” Mech Dev 120: 1351-1383). This transdifferentiation program is regulated by distinct pleitropically acting transcription factors such as Snail, Twists, Slug and Goosecoid (Thiery J. P., et al. (2006). “Complex networks orchestrate epithelial-mesenchymal transitions.” Nat Rev Mol Cell Biol 7: 131-142; LaBonne C., et al. (2000). “Snail-related transcriptional repressors are required in Xenopus for both the induction of the neural crest and its subsequent migration.” Dev Biol 221: 195-205).

At the biochemical level, the EMT program involves the downregulation of epithelial protein expression, notably cytokeratins and the induction of mesenchymal protein expression including vimentin, N-cadherin, fibronectin, platelet-derived growth factor receptor (PDGFR) and matrix metalloproteinases and acquisition of motility and invasiveness (Nieto Mass. (2002). “The snail superfamily of zinc-finger transcription factors.” Nat Rev Mol Cell Biol 3, 155-166). “Complex networks orchestrate epithelial-mesenchymal transitions.” Nat Rev Mol Cell Biol 7: 131-142). When active in cancer cells the EMT program enables these cells to complete the initial steps of invasiveness in the metastatic cascade, specifically local invasion, intravasation, survival in the circulation and extravasation.

Snail belongs to the Snail superfamily of zinc-finger transcription factors including Twist and SIP1, all considered essential for the induction of EMT in tumor metastasis. Snail was initially identified to play a role in embryonic development, neural differentiation, cell division and cell survival. Snail triggers the induction of metastasis during tumor progression by promoting the acquisition of invasive and migratory properties by tumor cells via transcriptional repression of metastasis suppressor genes such as E-cadherin (Peinado H, et al. (2004). “Snail mediates E-cadherin repression by the recruitment of the Sin3A/histone deacetylase 1 (HDAC1)/HDAC2 complex.” Mol Cell Biol 24: 306-319). Snail is transcriptionally regulated by NF-κB (Barbera M J, et al. (2004). “Regulation of Snail transcription during epithelial to mesenchymal transition of tumor cells.” Oncogene 23: 7345-7354; Julien S, et al. (2007). “Activation of NF-kappaB by Akt upregulates Snail expression and induces epithelium mesenchyme transition.” Oncogene 26: 7445-7456.) and post-transcriptionally by GSK-30-mediated phosphorylation that results in its cytoplasmic localization and proteasome degradation (Dominguez D, et al. (2003). “Phosphorylation regulates the subcellular location and activity of the snail transcriptional repressor.” Mol Cell Biol 23: 5078-5089; Zhou B P, et al. (2004). “Dual regulation of Snail by GSK-3beta-mediated phosphorylation in control of epithelial-mesenchymal transition.” Nat Cell Biol 6: 931-940).

In addition to E-cadherin, Snail downregulates the expression of tight junctions components, such as claudins and occludins and epithelial markers mucin-1 and cytokeratin 18. It also increases the expression of the mesenchymal markers vimentin and fibronectin, proteins involved in cancer invasion such as metalloproteinases 2 and 9 and transcription factors ZEB-1 and LEF-1 (Cano A, et al. (2000) “The transcription factor snail controls epithelial-mesenchymal transitions by repressing E-cadherin expression.” Nat Cell Biol 2: 76-83”; De Craene B, et al. (2005). “Unraveling signalling cascades for the Snail family of transcription factors.” Cell Signal 17: 535-547).

Among the survival pathways known to be associated with tumor progression and metastasis are the Raf-1/MEK/ERK and NF-κB pathways (Inoue J, et al. (2007). “NF-kappaB activation in development and progression of cancer.” Cancer Sci 98: 268-274; Granovsky A E, et al. (2008). “Raf kinase inhibitory protein: a signal transduction modulator and metastasis suppressor.” Cell Res 18: 452-457). Raf kinase inhibitor protein (RKIP) is a member of a conserved group of proteins called PEBP (phosphatidylethanolamine-binding protein) which participate in the regulation of growth and survival signaling pathways (Odabaei G, et al. (2004). “Raf-1 kinase inhibitor protein: structure, function, regulation of cell signaling, and pivotal role in apoptosis.” Adv Cancer Res 91: 169-200). RKIP has been reported to function by inhibiting the proliferative and survival Raf-1/MEK/ERK and NF-κB signaling pathways (Yeung K, et al. (1999). “Suppression of Raf-1 kinase activity and MAP kinase signalling by RKIP.” Nature 401: 173-177; Yeung K C, et al. (2001). “Raf kinase inhibitor protein interacts with NF-kappaB-inducing kinase and TAK1 and inhibits NF-kappaB activation.” Mol Cell Biol 21: 7207-7217). The importance of RKIP in metastases was demonstrated by the finding that the restoration of RKIP expression inhibits prostate cancer metastases in a murine model and hence, RKIP was identified as a metastasis suppressor protein (Fu Z, et al. (2006). “Metastasis suppressor gene Raf kinase inhibitor protein (RKIP) is a novel prognostic marker in prostate cancer.” Prostate 66: 248-256; Fu Z. et al. (2003). “Effects of raf kinase inhibitor protein expression on suppression of prostate cancer metastasis.” J Natl Cancer Inst 95: 878-889). RKIP was recently found to be under the regulation of the transcription repressor Snail (Beach S. et al. (2008). “Snail is a repressor of RKIP transcription in metastatic prostate cancer cells.” Oncogene 27: 2243-2248). RKIP expression levels correlated inversely with Snail expression in metastatic prostate samples, while overexpressing Snail in breast or prostate cell lines down-regulated RKIP expression. Metastasis is regulated by several complex mechanisms, though, it has been reported that both RKIP and Snail levels of expression in tumor cells dictate the metastatic behavior of tumor cells.

The proteasome has been implicated to play a major role in the pathogenesis and survival of cancers, mainly, through regulation of NF-κB activity and of anti-apoptotic gene products. The proteasome has also been involved in the inhibition of drug-induced apoptosis in tumor cells and in the development of drug resistance. Hence, proteasome inhibitors have been developed to target the proteasome with the objective to induce cancer cell cytostasis or cell death when used alone or in combination with other cytotoxics (Sterz J, et al. (2008). “The potential of proteasome inhibitors in cancer therapy.” Expert Opin Investig Drugs 17: 879-895).

Data provided herein show that NPI-0052 inhibits EMT in non-treated tumor cells. In particular, data also demonstrate that NPI-0052 downregulates mesenchymal and invasive markers such as vimentin and fibronectin and reverses the cell phenotype by inducing a mesenchymal to epithelial transition (MET) a process associated with the re-expression of E-cadherin. Concomitant with these observations are the findings derived herein of the in vitro tumor cell invasion and migration studies showing that NPI-0052 potentiates significantly the inhibition of both native and TNF-α-induced tumor cells migratory and invasive properties. RKIP loss or depletion has been associated with metastatic disease in an increasing number of solid tumors. Initially RKIP loss was identified as a prognostic marker for prostate cancer (Fu Z, et al. (2006). “Metastasis suppressor gene Raf kinase inhibitor protein (RKIP) is a novel prognostic marker in prostate cancer.” Prostate 66: 248-256; Fu Z, et al. (2003). “Effects of raf kinase inhibitor protein expression on suppression of prostate cancer metastasis.” J Natl Cancer Inst 95: 878-889). Further studies showed that RKIP is depleted in distant metastases for various tumor types, including colorectal and breast carcinoma (Minoo P, et al. (2007). “Loss of raf-1 kinase inhibitor protein expression is associated with tumor progression and metastasis in colorectal cancer.” Am J Clin Pathol 127: 820-827; Hagan S, et al. (2005). “Reduction of Raf-1 kinase inhibitor protein expression correlates with breast cancer metastasis.” Clin Cancer Res 11: 7392-7397) and could be a prognostic marker for disease-free survival. The anti-metastatic properties of RKIP have been attributed to its ability to inhibit survival pathways such as the Raf-1/MERK/ERK and NF-κB pathways (Yeung K, et al. (1999). “Suppression of Raf-1 kinase activity and MAP kinase signalling by RKIP.” Nature 401: 173-177; Yeung K C, et al. (2001). “Raf kinase inhibitor protein interacts with NF-kappaB-inducing kinase and TAK1 and inhibits NF-kappaB activation.” Mol Cell Biol 21: 7207-7217).

NPI-0052 was found to increase both RKIP mRNA and protein levels. Also, RKIP overexpression resulted in the inhibition of EMT-related gene products such as vimentin and fibronectin that are overexpressed in the metastatic DU-145 cell line and the re-appearance of epithelial gene products related to metastasis suppression including E-cadherin and cytokeratin 18. The above gene modifications were accompanied with tumor cell morphological changes associated with the acquisition of an epithelial cell phenotype.

The direct involvement of NF-κB inhibition by NPI-0052 in the modulation of Snail and RKIP expression was tested using the NF-κB chemical inhibitor DHMEQ, which inhibits the translocation of NF-κB from cytoplasm to the nucleus (Katsman A, et al. (2007). “Reversal of resistance to cytotoxic cancer therapies: DHMEQ as a chemo-sensitizing and immuno-sensitizing agent.” Drug Resist Updat 10:1-12). Data provided herein showed that DHMEQ was able to suppress significantly Snail expression and to induce RKIP suggesting that NF-κB inhibition has a leading role in the modulation of the above gene products and, concomitantly, in the regulation of invasion and metastasis.

In the metastatic prostate cell line DU-145, Snail transcription factor functions not only as a transcriptional repressor of E-cadherin but also of RKIP, while in primary and metastatic prostate tumors Snail expression is inversely correlated with RKIP and E-cadherin levels (Beach S, et al. (2008). “Snail is a repressor of RKIP transcription in metastatic prostate cancer cells.” Oncogene 27: 2243-2248). These observations suggested that Snail might be an appropriate therapeutic target to inhibit the EMT process and, in turn, to block tumor invasion.

Activation of NF-κB by upregulation of AKT results in downstream upregulation of Snail expression leading to induction of EMT suggesting that the Snail promoter is regulated positively by NF-κB p65 (Julien S, et al. (2007). “Activation of NF-kappaB by Akt upregulates Snail expression and induces epithelium mesenchyme transition.” Oncogene 26: 7445-7456). Data described herein showed that NPI-0052 has a significant inhibitory effect on Snail expression and this inhibition was directly associated with re-expression of RKIP and E-cadherin, inhibition of mesenchymal gene markers and induction of an epithelial cell phenotype as assessed by Snail silencing. These results suggested that inhibition of Snail may be pivotal for NPI-0052-mediated RKIP and E-cadherin induction and inhibition of EMT.

Overexpression of snail contributes directly to EMT, accelerating tumor survival, migration and bad prognosis, in cancer cell lines and tumor biopsies including breast cancer, gastric cancer, hepatocellular carcinomas, ovarian carcinoma, oral squamous cell carcinoma, and head and neck cancer (Blanco M J, et al. (2002) “Correlation of Snail expression with histological grade and lymph node status in breast carcinomas.” Oncogene 21: 3241-3246; Rosivatz E, et al. (2002). “Differential expression of the epithelial-mesenchymal transition regulators snail, SIP1, and twist in gastric cancer.” Am J Pathol 161: 1881-1891; Jiao W, et al. (2002). “Inverse correlation between E-cadherin and Snail expression in hepatocellular carcinoma cell lines in vitro and in vivo.” Br J Cancer 86: 98-101; Elloul S, et al. (2006). “Expression of E-cadherin transcriptional regulators in ovarian carcinoma.” Virchows Arch 449: 520-528; Yokoyama K, et al. (2001). “Reverse correlation of E-cadherin and snail expression in oral squamous cell carcinoma cells in vitro.” Oral Oncol 37: 65-71; Yang M H, et al. (2007). “Overexpression of NBS1 induces epithelial-mesenchymal transition and co-expression of NBS1 and Snail predicts metastasis of head and neck cancer.” Oncogene 26: 1459-1467). Data described herein showed that overexpression of the stable Snail form, Snail-6SA, in the non-metastatic prostate cell line LNCaP resulted in acquisition of the EMT phenotype with expression of vimentin and fibronectin. Other studies demonstrate Snail transfectants downregulate epithelial markers such as E-cadherin, mucin1, and cytokeratin 18, and upregulate and redistribute mesenchymal markers such as vimentin and fibronectin (Cano A, et al. (2000). “The transcription factor snail controls epithelial-mesenchymal transitions by repressing E-cadherin expression.” Nat Cell Biol 2: 76-83). In contrast, the shift of EMT markers and consequently the EMT phenotype was reversed by siRNA-mediated repression of Snail expression in DU-145.

NPI-0052 activity in tumor cell models may be mainly involved in induction of apoptosis in combination with other cytotoxics by mechanisms involving NF-κB downregulation. The anti-tumor properties of NPI-0052 have been evaluated in a wide range of non clinical studies including in vitro and in vivo models for a wide range of solid tumors and hematologic malignancies (Chauhan D, et al. (2005). “A novel orally active proteasome inhibitor induces apoptosis in multiple myeloma cells with mechanisms distinct from Bortezomib.” Cancer Cell 8: 407-419; Chauhan D, et al. (2008). “Combination of proteasome inhibitors bortezomib and NPI-0052 trigger in vivo synergistic cytotoxicity in multiple myeloma.” Blood 111: 1654-1664; Cusack J C, Jr., et al. (2006). “NPI-0052 enhances tumoricidal response to conventional cancer therapy in a colon cancer model.” Clin Cancer Res 12: 6758-6764; Miller C P, et al. (2007). “NPI-0052, a novel proteasome inhibitor, induces caspase-8 and ROS-dependent apoptosis alone and in combination with HDAC inhibitors in leukemia cells.” Blood 110: 267-277; Ruiz S, et al. (2006). “The proteasome inhibitor NPI-0052 is a more effective inducer of apoptosis than bortezomib in lymphocytes from patients with chronic lymphocytic leukemia.” Mol Cancer Ther 5: 1836-1843). NPI-0052 as single agent has shown advantages compared to conventional proteosome inhibitors such as Bortezomib and MG-132 with regards to higher speed and duration of action, wider spectrum of inhibitory effects to the 20S proteasome, greater suppressive effect on NF-κB activation in many tumor cell models, potent apoptotic activity at low concentrations and ability to reverse tumor resistance to Bortezomib in vitro and in vivo (Chauhan D, et al. (2006). “A novel proteasome inhibitor NPI-0052 as an anticancer therapy.” Br J Cancer 95: 961-965; Chauhan D, et al. (2008). “Combination of proteasome inhibitors bortezomib and NPI-0052 trigger in vivo synergistic cytotoxicity in multiple myeloma.” Blood 111: 1654-1664). In addition, NPI-0052 as single agent has good tolerance in vivo in mice models and limited toxicity in human colony-forming assays (Baritaki S, et al. (2008). “Inhibition of Yin Yang 1-dependent repressor activity of DR5 transcription and expression by the novel proteasome inhibitor NPI-0052 contributes to its TRAIL-enhanced apoptosis in cancer cells.” Immunol 180: 6199-6210; Chauhan D, et al. (2005). “A novel orally active proteasome inhibitor induces apoptosis in multiple myeloma cells with mechanisms distinct from Bortezomib.” Cancer Cell 8: 407-419). As described herein, NPI-0052 is able to reverse prostate tumor cell resistance to TRAIL apoptosis with a mechanism involving induction of TRAIL receptors (DR4 and DR5) and activation of type I and type II apoptotic pathways via Ying Yang 1 (YY1) and NF-κB inhibition (see also, Baritaki S, et al. (2008). “Inhibition of Yin Yang 1-dependent repressor activity of DR5 transcription and expression by the novel proteasome inhibitor NPI-0052 contributes to its TRAIL-enhanced apoptosis in cancer cells.” Immunol 180: 6199-6210, incorporated by reference in its entirety). Thus, NPI-0052 is an effective anti cancer-agent with unique pharmacogenic properties that can achieve high levels of proteasome inhibition in vitro and in vivo.

In summary, data described herein demonstrate that NPI-0052 inhibited Snail transcription and expression in the metastatic prostate cancer cell line DU-145. NPI-0052-induced Snail suppression correlated with NPI-0052-induced RKIP expression, and NPI-0052 induced repression of Snail is due, in part, to NPI-0052-mediated inhibition of NF-κB activity. Also, a specific NF-κB inhibitor, DHMEQ, mimicked NPI-0052-induced repression of Snail and induction of RKIP, and NPI-0052-induced repression of Snail resulted in the inhibition of EMT and induction of E-cadherin. Treatment with siRNA against Snail mimic NPI-0052-mediated effects and resulted in the inhibition of EMT in the metastatic DU-145 cell line. In addition, overexpression of Snail induced EMT in the non-metastatic LNCaP prostate cancer cell line, and overexpression of RKIP mimicked NPI-0052-induced inhibition of Snail and EMT in the metastatic DU-145 cell line.

The data suggest a novel function and therapeutic application for the proteasome inhibitor NPI-0052 in the management of metastatic prostate tumors and other cancers. The clinical relevance and significance of inhibiting Snail and restoring RKIP expression by NPI-0052 is likely to correlate with a favorable clinical outcome accompanied by diminution of tumor progression and spread. Moreover, RKIP induction by NPI-0052 may improve the efficacy of anti-tumor therapies, especially if they are combined with conventional immuno- and/or or chemo-therapeutics, as well as the host immune surveillance against cancer (Baritaki S, et al. (2007). “Regulation of tumor cell sensitivity to TRAIL-induced apoptosis by the metastatic suppressor Raf kinase inhibitor protein via Yin Yang 1 inhibition and death receptor 5 up-regulation.” J Immunol 179: 5441-5453). In addition, RKIP and Snail expression profiles in tumors may be used as potential prognostic biomarkers.

### EXAMPLES

### Example 1

### Materials and Methods

Materials and methods used in the examples described herein are well known in the art. Some methods are described in Baritaki S, et al. (2008). “Inhibition of Yin Yang 1-dependent repressor activity of DR5 transcription and expression by the novel proteasome inhibitor NPI-0052 contributes to its TRAIL-enhanced apoptosis in cancer cells.” Immunol 180: 6199-6210; Baritaki S, et al., (2007). “Regulation of tumor cell sensitivity to TRAIL-induced apoptosis by the metastatic suppressor Raf kinase inhibitor protein via Yin Yang 1 inhibition and death receptor 5 up-regulation.” J Immunol 179: 5441-5453; Beach S, et al. (2008). “Snail is a repressor of RKIP transcription in metastatic prostate cancer cells.” Oncogene 27: 2243-2248, incorporated by reference in their entireties.

Cell lines included the prostate carcinoma cell lines PC-3, DU-145 (both metastatic bone-derived human androgen-independent human prostatic adenocarcinoma; PC-3 have a high mestastatic potential compared to DU-145 which have a moderate metastatic potential) and LNCaP (non-metastatic bone-derived human androgen dependent human prostatic adenocarcinoma), and Ramos (Burkitt's lymphoma cell line).

NF-κB activity was determined using an NF-κB-Luciferase reporter plasmid (pNF-κBLuc) purchased from Invitrogen (Carlsbad, Calif.). A CMV-HARKIP expression vector containing the full-length cDNA of RKIP under control of a CMV promoter was used for ectopic RKIP expression (Chatterjee D. et al. (2004) “RKIP sensitizes prostate and breast cancer cells to drug-induced apoptosis.” J Biol Chem 279: 17515-17523, incorporated by reference in its entirety). A CMV-flag-Snail expression construct (CMV-fsnail) was used for Snail overexpression in LNCaP cells. A mutated stable variant (CMV-fsnail 6SA) containing all six phosphorylated serine residues in the consensus GSK-3β sites mutated to alanine was used to circumvent the possible effect of the highly unstable Snail on RKIP expression (Beach S. et al. (2008). “Snail is a repressor of RKIP transcription in metastatic prostate cancer cells.” Oncogene 27: 2243-2248, incorporated by reference in its entirety).

### Example 2

### Reversal of Tumor Cell Resistance to Chemotherapy by NPI-0052

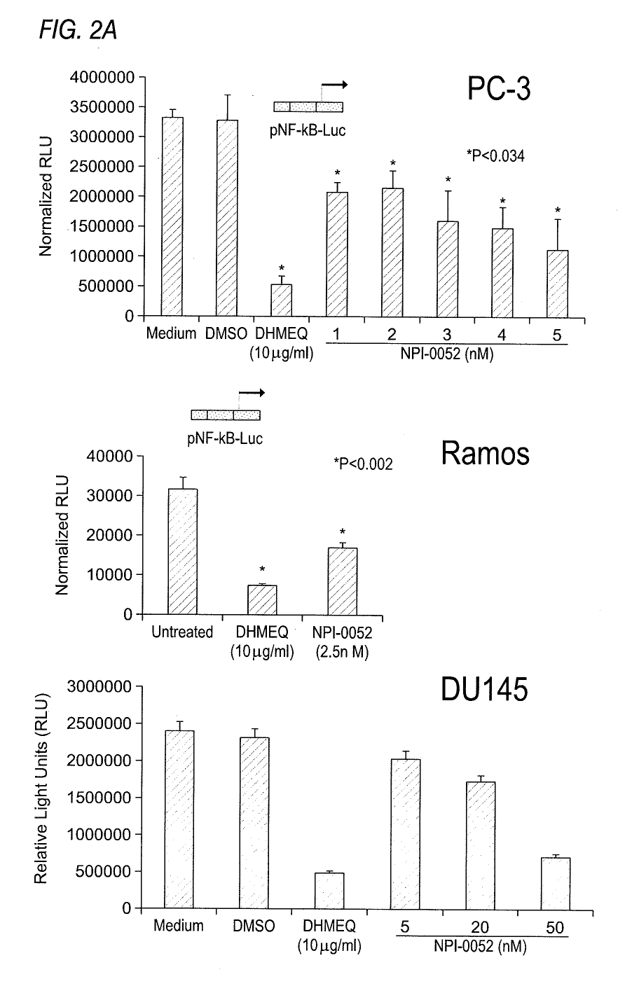

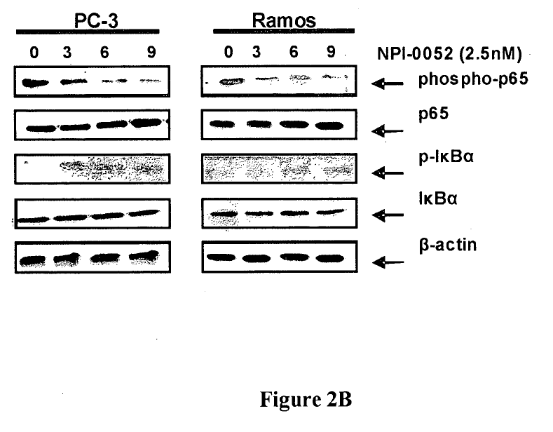

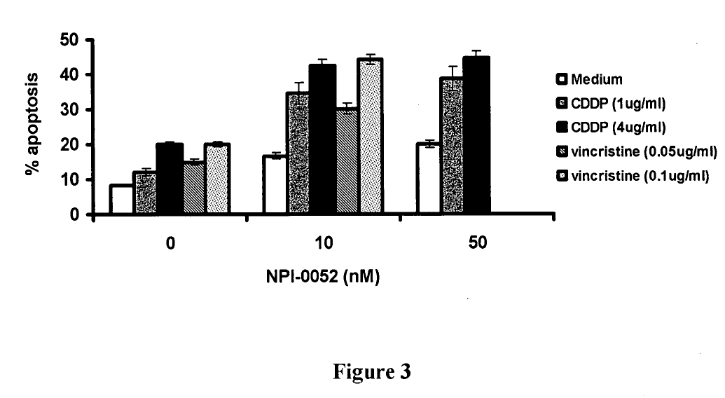

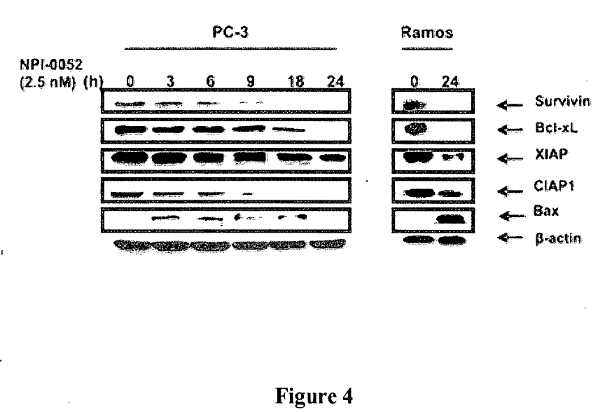

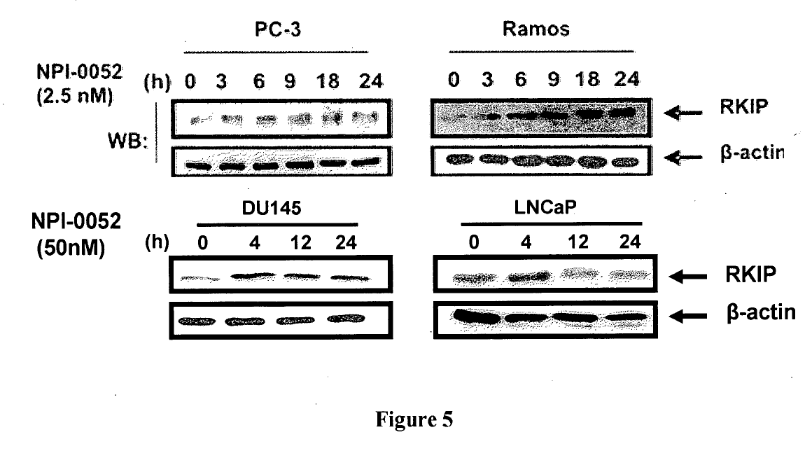

A series of experiments were carried out to investigate whether NPI-0052, is able to reverse tumor resistance to chemotherapy. FIG. 2A shows graphs of luciferase activity in PC-3 cells, Ramos cells, and DU-145 cells. Cells were transfected with NFκB-luciferase reporter constructs and treated with NPI-0052, DHMEQ, DMSO, medium only. FIG. 2B shows Western blots of transfected PC-3 and transfected Ramos cells for NF-κB (p65), phosphorylated p65, nuclear factor of kappa light polypeptide gene enhancer in B-cells inhibitor-alpha (IkBα), and phosphorylated IkBα. pIkbα accumulates in tumor cells treated with NPI-0052. NPI-0052 inhibits expression of luciferase from a reporter construct containing the NF-κB promoter. FIG. 3 shows a graph of percentage apoptosis with increasing concentrations of NPI-0052 in DU-145 cells treated with cis-diamminedichloridoplatinum(II) (CDDP) or vincristine. NPI-0052 sensitizes tumor cells to CDDP and vincristine-mediated apoptosis. FIG. 4 shows Western blots of PC-3 cells and Ramos cells treated with 2.5 nM NPI-0052. NPI-0052 modulates expression anti-apoptotic gene products involved in chemoresistance, including Survivin, Bcl-xL, XIAP, CIAPI, and Bax. FIG. 5 shows Western blots of PC-3 and Ramos cells treated with 2.5 nM NPI-0052, and DU145 and LNCaP cells treated with 50 nM NPI-0052, where RKIP expression is induced by NPI-0052.

From these data, NPI-0052 sensitizes tumor cells to drug-induced apoptosis via inhibition of NF-κB. NPI-002-mediated inhibition of NF-κB results in inhibition of NF-κB-regulated anti-apoptotic gene products (e.g. Bcl-xL, cIAPs, XIAP) and induction of apoptosis. NPI-0052 upregulates RKIP which acts as an additional inhibitor of NF-κB. In addition, RKIP is directly involved in NPI-0052-induced tumor cell sensitization to drug-mediated apoptosis.

### Example 3

### Reversal of Tumor Cell Resistance to TRAIL-Mediated Apoptosis by NPI-0052

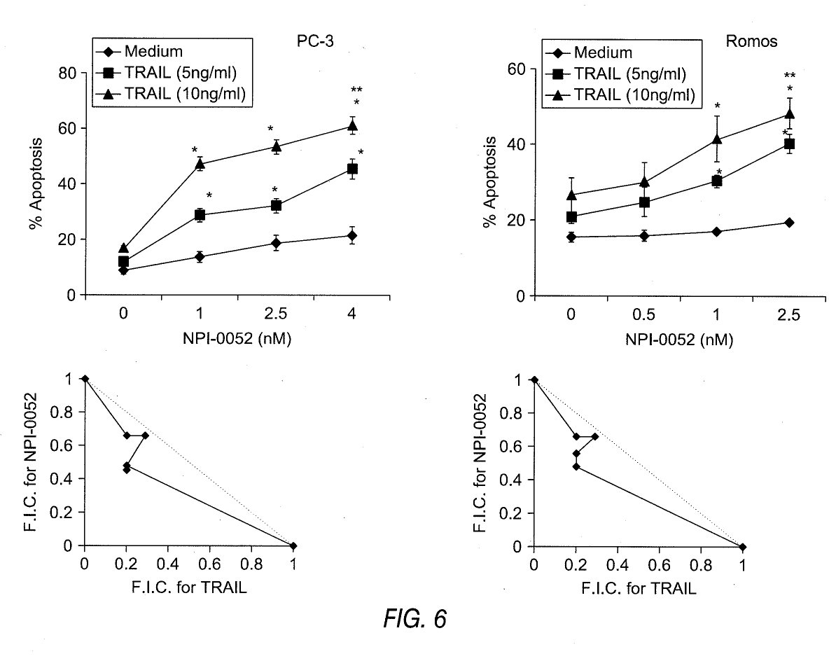

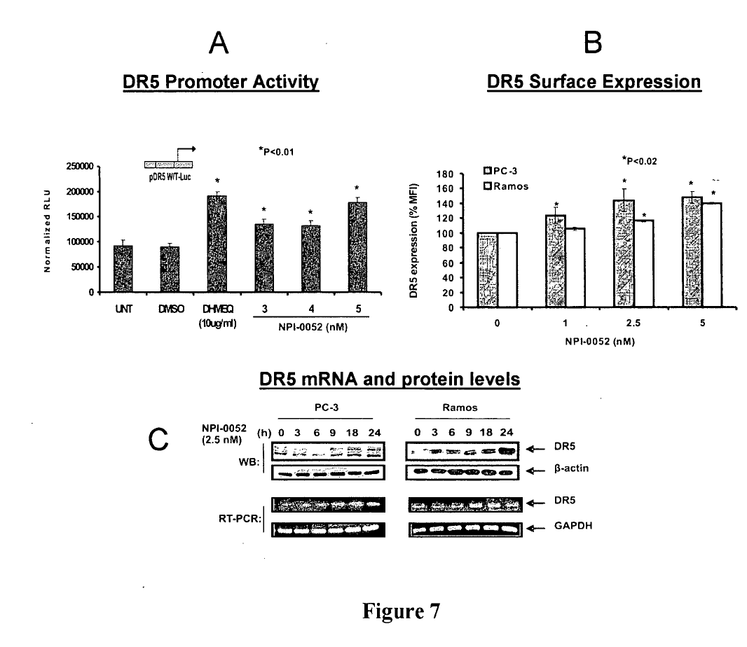

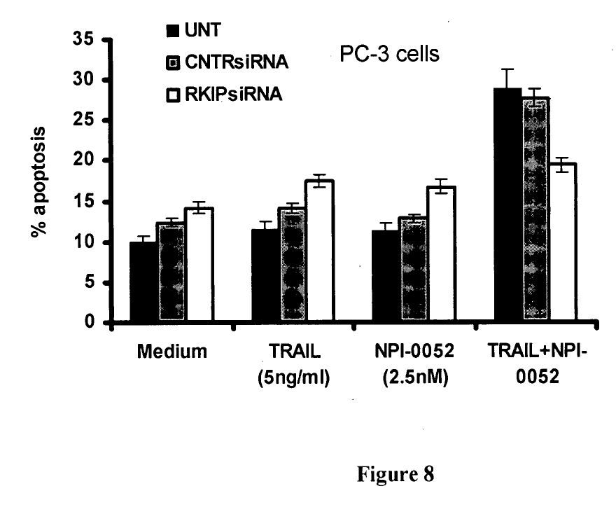

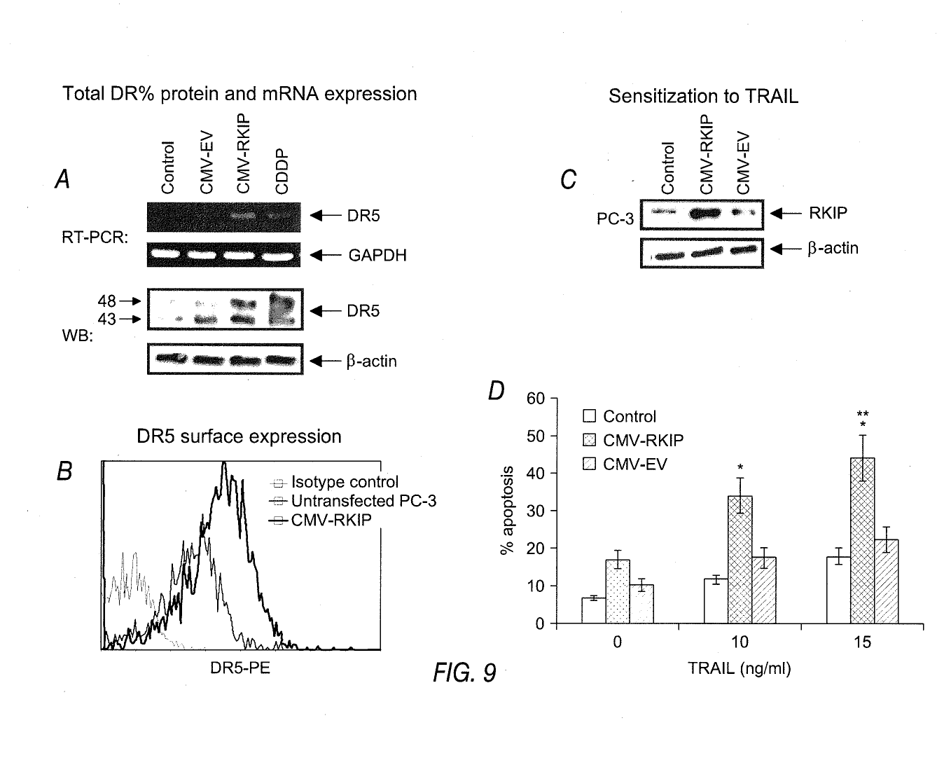

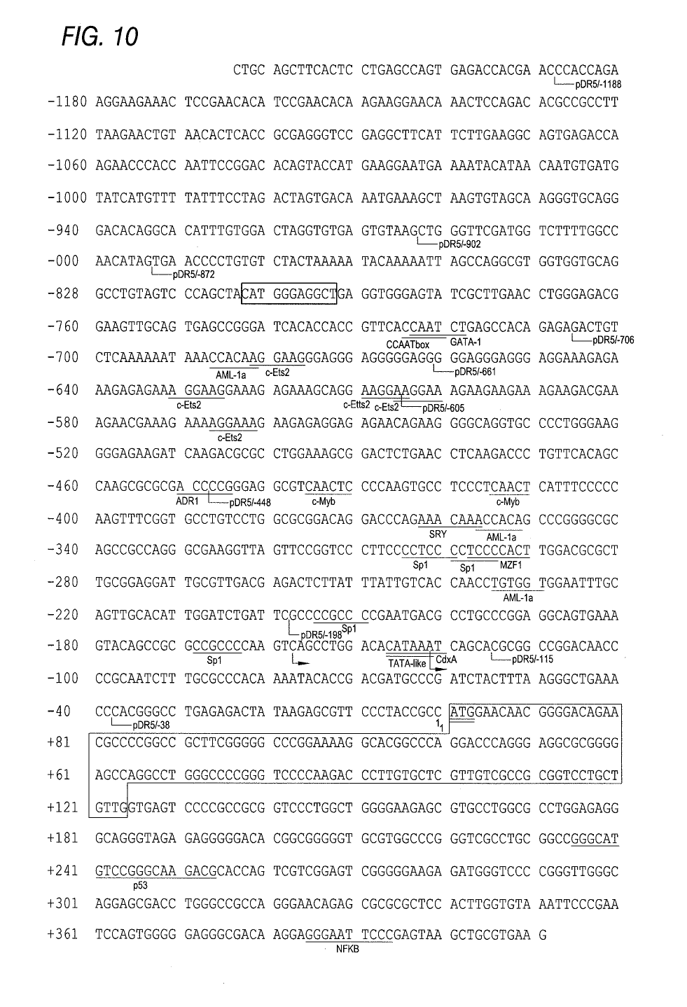

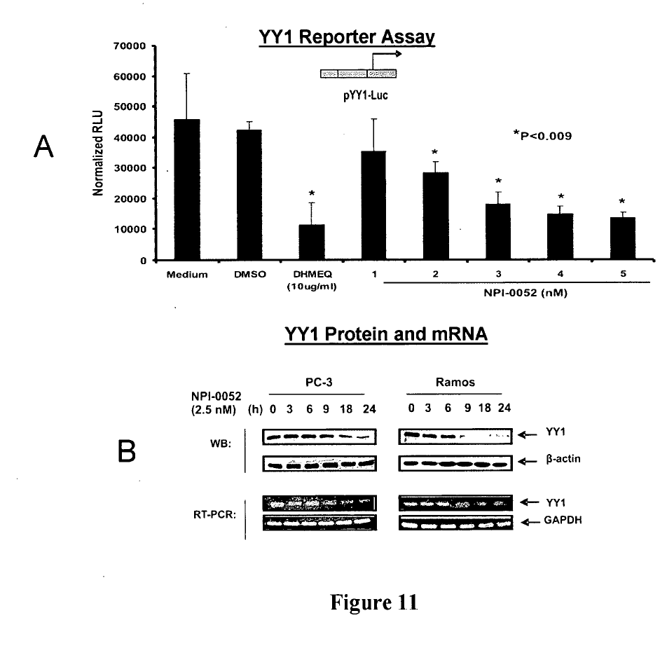

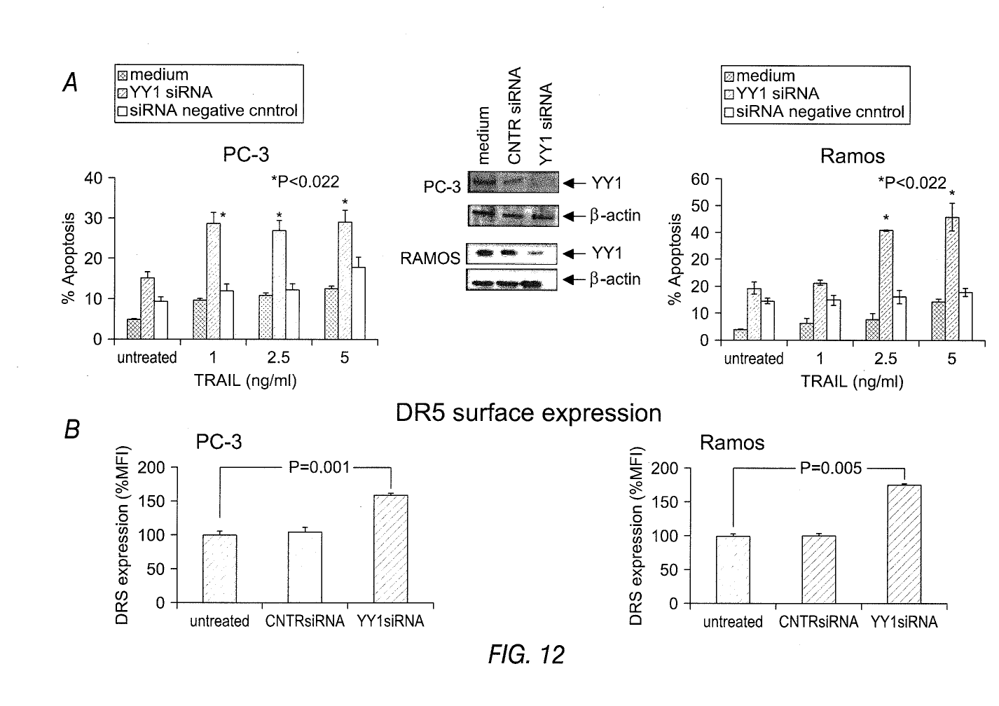

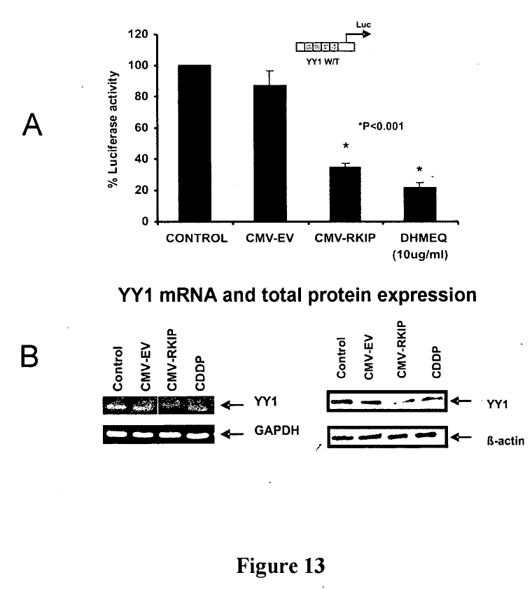

TRAIL exhibits potent tumoricidal activity in a variety of human cancer cell lines in vitro and in vivo in several xenograft tumor models with minimal or no toxicity to nonmalignant human cells. TRAIL is produced by immune cells, and is a ligand for death receptors DR4, DR5, DcR1, and DcR2. To investigate reversal of tumor cell resistance to TRAIL-mediated apoptosis by NPI-0052, tumor cells were treated with TRAIL and NPI-0052. FIG. 6 shows graphs of percentage apoptosis with increasing concentrations of NPI-0052 in PC-3 and Ramos cells treated with 5 ng/ml or 10 ng/ml Tumor necrosis (TNF)-related apoptosis-inducing ligand (TRAIL). NPI-0052 sensitizes tumor cells to TRAIL-mediated apoptosis. FIG. 7A shows a graph of luciferase activity in cells transfected with a DR5-luciferase reporter construct and treated with 10 μg/ml DHMEQ, or 3 mM, 4 mM, or 5 nM NPI-0052. Controls included untransfected (UNT) and DMSO. FIG. 7B shows a graph of DR5 expression in PC-3 and Ramos cells treated with 1 nM, 2.5 nM, or 5 nM NPI-0052. FIG. 7C shows Western blots and RT-PCR gels of PC-3 or Ramos cells treated with 2.5 nM NPI-0052. NPI-0052 upregulates DR5 transcription and expression. FIG. 8 shows a graph of percentage apoptosis in PC-3 cells transfected with CNTR siRNA or RKIP siRNA, and treated with 5 ng/ml TRAIL, 2.5 nM NPI-0052, or 5 ng/ml TRAIL and 2.5 nM NPI-0052. RKIP may have a direct role in the NPI-0052-induced sensitization to TRAIL. FIG. 9A shows a RT-PCR gel and Western blot of PC-3 cells transfected with CMV-EV or CMV-RKIP expression constructs, or treated with CDDP. FIG. 9B shows a graph of DR5 surface expression in PC-3 cells transfected with CMV-RKIP expression constructs. FIG. 9C shows a Western blot of PC-3 cells transfected with CMV-RKIP or CMV-EV expression constructs. FIG. 9D shows percentage apoptosis in cells transfected with CMV-RKIP or CMV-EV expression constructs, and treated with 0 ng/ml, 10 ng/ml, or 15 ng/ml TRAIL. RKIP overexpression results in DR5 upregulation and cell sensitization to TRAIL-mediated apoptosis. YY1 acts as a suppressor of DR5 transcription and is positively regulated by NF-κB. FIG. 10 shows the sequence of the DR5 promoter. FIG. 11A shows a graph of luciferase activity in cells transfected with a pYY1-luciferase reporter construct, and treated with DHMEQ or 1 nM, 2 nM, 3 nM, 4 nM, or 5 nM NPI-0052. FIG. 11B shows Western blots and RT-PCR gels of PC-3 cells or Ramos cells treated with 2.5 nM NPI-0052. NPI-0052 inhibits expression from the YY1 promoter, and reduces expression of the YY1 gene in PC-3 cells and Ramos cells. FIG. 12A shows graphs of percentage apoptosis in PC-3 and Ramos cells transfected with YY1 siRNA and treated with 1 ng/ml, 2.5 ng/ml, or 5 ng/ml TRAIL. Also shown are Western blots of PC-3 and Ramos cells transfected with YY1 siRNA. FIG. 12B shows graphs of DR5 expression in PC-3 cells or Ramos cells transfected with YY1 siRNA and upregulation of DR5. Transfection controls include medium only and siRNA negative control. YY1 has a direct role in tumor sensitization to TRAIL apoptosis. FIG. 13A shows a graph of luciferase activity in cells transfected with a YY1-lucificerase reporter construct, and CMV-EV or CMV-RKIP expression constructs and CMV-RKIP inhibited YY1 luciferase activity. Controls include transfected with the YY1-lucificerase reporter construct only, and cells treated with 10 μg/ml DHMEQ only. FIG. 13B shows a PT-PCR gel and Western blot of cells transfected with a YY1-lucificerase reporter construct, and CMV-EV or CMV-RKIP expression constructs. CMV-RKIP inhibited both mRNA transcription and protein expression of YY1. YY1 is inhibited by RKIP overexpression via NF-κB down-regulation.

From these data, NPI-0052 sensitizes tumor cells to TRAIL apoptosis via upregulation of DR5 and inhibition of NF-κB. NPI-0052 upregulates DR5 via YY1 inhibition resulting from NF-κB inhibition. NPI-0052 upregulates RKIP which acts as an additional inhibitor of NF-κB. In addition, RKIP is directly involved in NPI-0052-induced tumor cell sensitization to TRAIL-mediated apoptosis.

### Example 4

### Inhibition of Both Snail and Initiation of Epithelial Mesenchymal Transition (EMT) and Metastasis by NPI-0052

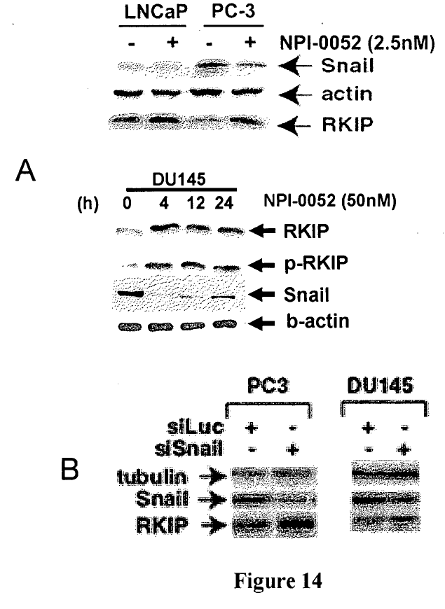

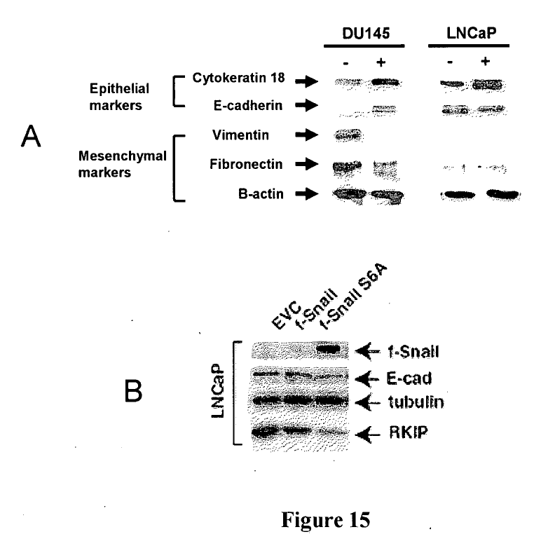

To investigate the role of NPI-0052 in the inhibition of Snail, initiation of EMT, and metastasis, cells were treated with NPI-0052. FIG. 14A shows Western blots of LNCaP cells, PC-3 cells, and DU145 cells treated with 0 nM, 2.5 nM, or 50 nM NPI-0052. FIG. 14B shows a Western blot of PC3 cells and DU145 cells transfected with siLuc or siSnail. There is a reverse correlation between RKIP and Snail expression. Treatment with Snail siRNA increases RKIP expression. FIG. 15A shows a Western blot of DU145 cells and LNCaP cells treated (+), or untreated (−) with NPI-0052. Treatment with NPI-0052-induced the expression of epithelial markers and inhibited mesenchymal markers. FIG. 15B shows a RT-PCR gel of LNCaP cells transfected with f-Snail or f-Snail S6A expression constructs. NPI-0052 modulates expression of EMT-related gene markers in DU-145 cells and LNCaP cells. Over-expression of Snail in LNCaP downregulates RKIP and E-cadherin.

From these data, Snail negatively regulates RKIP expression. NPI-0052 induces metastasis suppressor gene RKIP and downregulates Snail expression in metastatic and non-metastatic prostate cells. Inhibition of Snail by NPI-0052 or Snail siRNA in metastatic tumors results in induction of the metastasis suppressor genes RKIP and E-cadherin. In addition, NPI-0052 mediated Snail inhibition results in upregulation of epithelial gene markers (e.g. E-cadherin, cytokeratin 18) and downregulation of mesenchymal markers (e.g. vimentin) resulting in inhibition of EMT.

In summary, NPI-0052 sensitizes tumor cells to chemo- and TRAIL-mediated apoptosis via direct inhibition of NF-κB. NPI-0052 induces RKIP expression which acts as an additional inhibitor of NF-κB. RKIP is directly involved in tumor cell sensitivity to chemotherapeutic drugs or TRAIL. NF-κB inhibition by NPI-0052 and/or by NPI-0052-induced RKIP up-regulation result in inhibition of anti-apoptotic gene products and inhibition of YY1 resulting in induction of DR5 and sensitization to TRAIL. Over-expression of RKIP by NPI-0052 is reversely correlated with SNAIL down-regulation and inhibition of EMT-inducing gene products (e.g. vimentin, fibronectin) resulting in inhibition of metastasis.

### Example 5

### NPI-0052 Inhibits NF-κB Activation

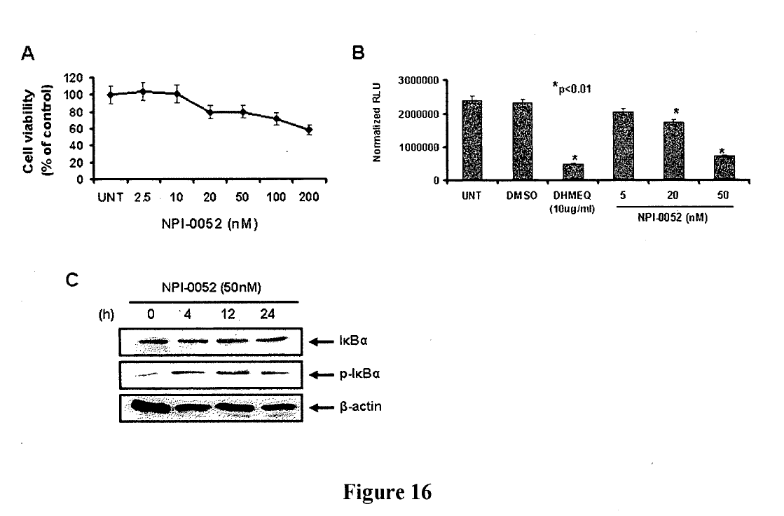

To confirm the inhibitory effect of NPI-0052 on NF-κB, the effect of NPI-0052 on different levels of NF-κB activation was monitored using NF-κB promoter activity and expression of phospho-IκBα. Subtoxic NPI-0052 concentrations were determined for DU-145 cells by performing drug titration using trypan blue exclusion assay (FIG. 16A). 50 nM NPI-0052 was optimized at a ID20 drug concentration. In the NF-κB-luciferase reporter system, increasing concentrations of NPI-0052 inhibited proportionally the NF-κB promoter activity as shown in FIG. 16B. In FIG. 16B, values represent the mean±SEM of three independent experiments and were calculated based on the control value set at 100% (control: untreated cells). Transfected cells treated with 10 μg/ml DHMEQ served as a positive inhibition control of the NF-κB promoter activity. P value: treated vs untreated cells (Mann-Whitney U test). RLU: relative light units. FIG. 16C shows that 50 nM of NPI-0052 induced time-dependent accumulation of phospho-IkBα protein due to lack of degradation by the proteasome, namely, NPI-0052 prevents p-IkBα degradation and results in its cytoplasmic accumulation. DU-145 cells were treated with 50 nM NPI-0052 for the indicated time points and Western blot analysis was performed with whole cell lysates for the detection of both phosphorylated and total IkBα levels. These findings demonstrate the suppressive effect of NPI-0052 on NF-κB activity in metastatic DU-145 cells, and the inhibition of gene products that activate NF-κB.

### Example 6

### NPI-0052 Suppresses the Expression of Mesenchymal Gene Products and Induces the Expression of Epithelial Gene Products in Prostate Tumor Cells

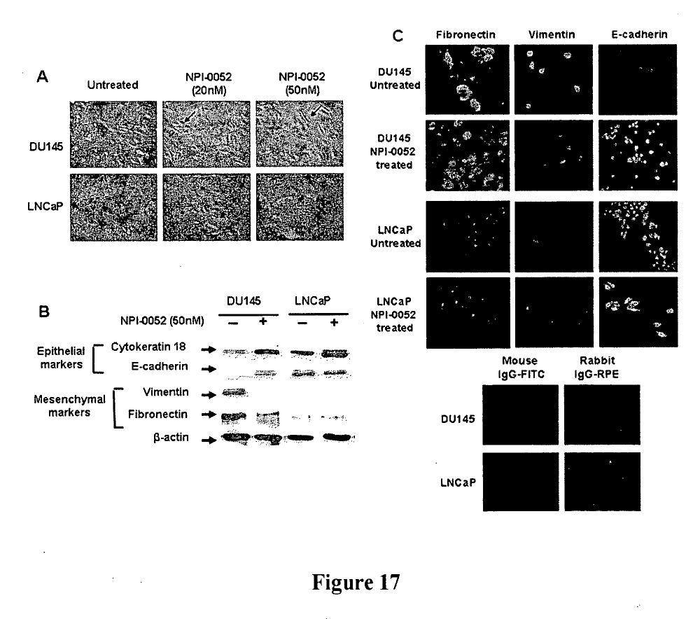

Since NF-κB plays a crucial role in the induction of metastasis and can be inhibited by NPI-0052, the ability of NPI-0052 to inhibit metastasis was investigated. Based on preliminary findings, it was hypothesized that NPI-0052 may affect the expression of gene products which determine the epithelial and mesenchymal cell phenotype and which are under the direct or indirect regulation of NF-κB. Such effects will result in changes of migratory and invasive tumor properties and inhibition of EMT. Indeed, NPI-0052 induced morphological changes in the metastatic cell line DU-145. Referring to FIG. 17A, after 24 h treatment with increasing concentrations of NPI-0052, DU-145 cells acquired a more epithelial-like phenotype (see arrows) accompanied with lower cell mobility. In contrast, the non-metastatic LNCaP cells treated with the same concentrations of NPI-0052 maintained the epithelial phenotype without any significant change.

The genetic background of the observed NPI-0052-induced cell morphological changes was examined by monitoring the expression patterns of epithelial markers including cytokeratin 18 and E-cadherin as well as the expression of the mesenchymal phenotype-related gene products fibronectin and vimentin. The protein levels were assessed by Western blot and immunofluoresence analysis in both the DU-145 and LNCaP cell lines prior to and following treatment with NPI-0052. Total protein lysates derived from DU-145 and LNCaP cells that were treated for 4 h with 50 nM NPI-0052 were subjected to Western blot analysis for determination of protein expression of the indicated epithelial and mesenchymal gene markers. The results were compared with the expression profiles of the same gene products in untreated cell lysates. Actin was used as an internal control for loading. NPI-0052 significantly induced the expression of E-cadherin and cytokeratin 18 mainly in DU-145 cells, where the basal levels of the above epithelial gene products are low (FIGS. 17B, 17C). In contrast, NPI-0052 downregulated the levels of fibronectin and blocked almost completely vimentin expression. LNCaP cells have undetectable vimentin expression and their low basal fibronectin level remained unchanged after NPI-0052 treatment (FIGS. 17B, 17C). These findings suggest that NPI-0052 modifies, at least at the post-transcriptional level, gene products involved in EMT induction and resulting in inhibition of the mesenchymal cell phenotype.

### Example 7

### NPI-0052 Suppresses the Migratory and Invasive Properties of the Metastatic DU-145 Cells

The functional significance of the NPI-0052-induced changes in the expression profiles of the above metastasis-related gene products was expected to be reflected in the migratory and invasive cell properties. To maximize the effects of NPI-0052 on cell migration and invasion activity, DU-145 cells were stimulated with TNF-α since TNF-α has been shown to induce the expression of many genes involved in tumor metastasis (van de Stolpe A, et al. (1994). “12-O-tetradecanoylphorbol-13-acetate- and tumor necrosis factor alpha-mediated induction of intercellular adhesion molecule-1 is inhibited by dexamethasone. Functional analysis of the human intercellular adhesion molecular-1 promoter.” J Biol Chem 269: 6185-6192, incorporated by reference in its entirety). To determine tumor cell migratory and invasive properties, the cells were seeded onto the top chamber of the appropriate plates with or without TNF-α in the presence or absence of NPI-0052 and then examined for migration and invasion.

In vitro cell invasion and migration assays were performed using the 24/well BD Biocoat 3 micron migration chambers or 8 micron Matrigel invasion chambers (Becton Dickinson Labware, Bedford, Mass.), respectively, according to manufacturer's instructions. For both assays, cells were starved for 4-5 h prior to setting up the assay by adding basal medium supplemented with 0.1% BSA. RPMI 1640 medium containing 10% serum was used as chemoatractant. For single drug treatment 50 nM NPI-0052 or 100 ng/ml TNF-α was added to both chambers. For combinational treatment NPI-0052 was added 4 h before the addition of TNF-α. Measurement of cell migration was performed by cell post-labeling with BD Calcein AM Fluorescent dye (Becton Dickinson Labware, Bedford, Mass.) at 4 ug/ml. Data were expressed as the fold cell migration derived from the mean RFU of cell migration through the membrane towards FBS divided by the mean RFU of cell migration in the absence of FBS. For the invasion assays, inserts allowing cell migration were used as controls (control inserts). Invaded cells through Matrigel were counted under the microscope at 40× magnification. Data were expressed as the percent invasion through the Matrigel Matrix and membrane relative to the migration through the control insert membrane.

For migratory assays, 10⁵ cells were seeded in the upper chamber, while in the lower chamber RPMI 1640 containing 10% FBS was added. Cell migration was allowed for 22 h in a humidified incubator at 37° C. with 5% CO₂ and measured in a fluorescence plate reader using a cell post-labeling approach with BD Calcein AM Fluorescent dye. Only labeled cells that had migrated through the pores of the membrane were detected. The data were expressed as the fold cell migration derived from the mean RFU of cell migration through the membrane towards FBS divided by the mean RFU of cell migration in the absence of FBS. For invasion assays, 2.5×10⁴ cells were seeded in the upper chamber, while RPMI 1640 containing 10% FBS was added in the lower chamber. The cells were treated or left untreated with combinations of 50 nM NPI-0052 and 100 ng/ml TNFα and incubated for 22 h. Invaded cells were counted under microscope after matrigel membrane fixation and staining with crystal violet. Data are expressed as the percent invasion through the Matrigel Matrix and membrane relative to the migration through the control insert membrane. For both assays P values were set significant at the level of 0.05 and were calculated using the Mann-Whitney U test.

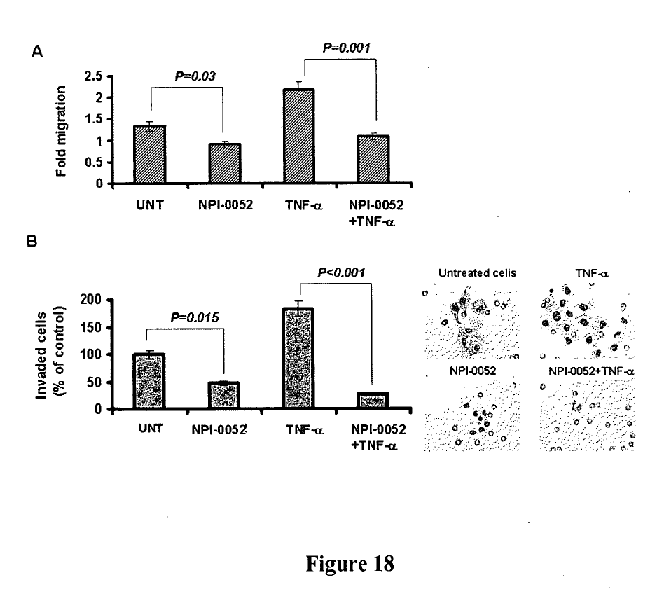

NPI-0052 suppressed significantly both the tumor cells migratory (FIG. 18A) and invasive (FIG. 18B) properties. FIG. 18B right panel shows representative pictures of invaded cells (blue staining) in different treatment conditions (40× magnification), where the untreated cells were the control. TNF-α induced tumor cell migration and invasion (augmented by almost 10-fold compared to untreated cells) was also suppressed by NPI-0052. These findings suggest that NPI-0052 can regulate metastasis via modification of EMT-related gene products and resulting in inhibition of tumor cell migratory and invasive properties.

### Example 8

### NPI-0052 Induces the Metastasis Suppressor Gene Product, RKIP, Whose Overexpression Reverses the Mesenchymal Cell Phenotype in DU-145 Cells

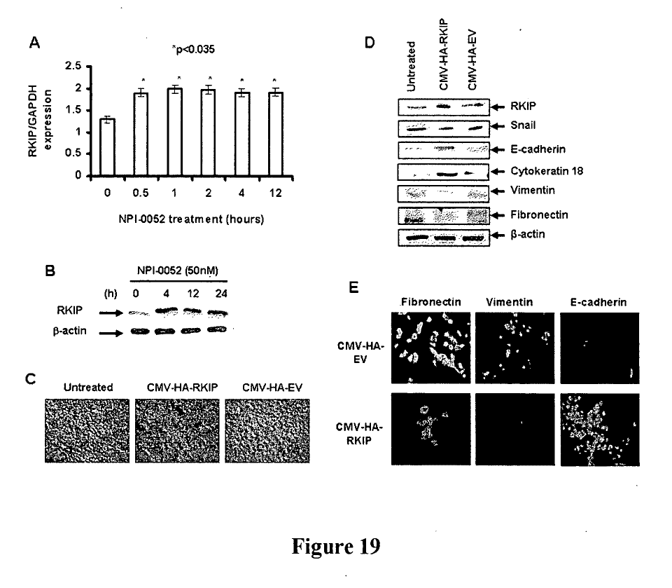

The Raf-1 Kinase inhibitor protein (RKIP) inhibits NF-κB suppression and suppresses metastasis. To determine whether NPI-0052 interferes with the transcriptional regulation of RKIP in DU-145 cells, RKIP expression in cells treated with 50 nM NPI-0052 for various time periods (0.5, 1, 2, 3, 4 and 12 h) was measured. RKIP transcript levels were determined by RT-PCR for each time point tested (0-12 h). Normalized mRNA values were derived by dividing the mRNA value of each target gene with the corresponding quantity of GAPDH mRNA. Statistical analysis was performed using the Mann-Whitney U test. P values: treated vs untreated cells. The data represent the mean values±SEM from three independent experiments. RKIP mRNA expression showed a constant increase starting at 30 min post-treatment up to the final time point of 12 h (FIG. 19A). Protein levels were also determined. Protein lysates were harvested at various time points and subjected to Western Blot analysis for RKIP protein. Actin was used as an internal control for loading. Blots are representative of three independent and reproducible experiments. RKIP protein levels followed a time-dependent significant increase in the presence of NPI-0052 and starting as early as 4 h post-treatment (FIG. 19B).

To determine the direct effect of RKIP induction in the regulation of the metastatic phenotype of DU-145 cells, RKIP was ectopically expressed in DU-145 cells using a CMV-HA-RKIP expression vector. Cells over-expressing RKIP were characterized by morphological changes related to a more epithelial like phenotype as shown in FIG. 19C.

Morphological changes were further reflected in changes in gene expression. Total cell lysates were harvested from DU-145 cells after over-expression of RKIP for 48 h using a CMV-HA-RKIP vector and subjected to Western Blot analysis. For IFA, DU145 cells transfected with the CMV-HA-RKIP or CMV-HA-EV constructs cells were stained with rabbit anti-fibronectin, rabbit anti-vimentin and mouse anti-E-cadherin. Referring to FIG. 19D and FIG. 19E, over-expression of RKIP induces the expression of metastasis suppressor gene E-cadherin as well as the expression of the epithelial gene marker cytokeratin 18. In contrast RKIP induction downregulates the mesenchymal gene products, such as vimentin and fibronectin. A CMV empty vector (CMV-EV) was used as a control. Blots are representative of three independent and reproducible experiments.

Cells transfected with the control CMV empty vector (CMV-HA-EV) did not show any significant differences in the expression profiles of the gene products tested above compared to untransfected cells. RKIP overexpression also resulted in decrease of Snail, an important EMT-inducer. These above findings suggested that the induction of the metastasis suppressor gene product, RKIP is a crucial factor underlying the mechanism by which NPI-0052 regulates metastasis.

### Example 9

### NPI-0052 Downregulates Snail Expression Via NF-κB Inhibition and Resulting in RKIP Upregulation and Inhibition of EMT

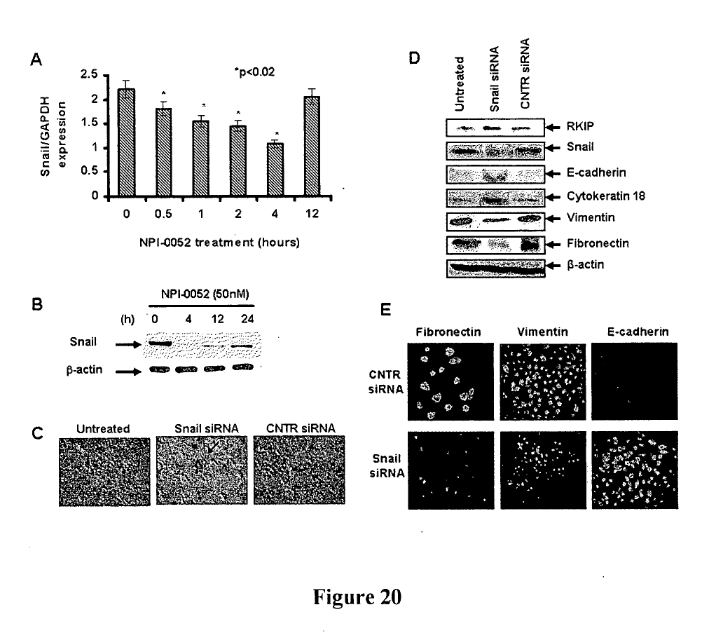

RKIP is under the negative transcriptional regulation of Snail, while Snail has been previously reported to be positively regulated, in part, by NF-κB (Beach et al., 2008; Barbera et al., 2004). Treatment of DU-145 cells with 50 nM of NPI-0052 induced a potent decrease in Snail transcription as early as 1 h post-treatment and reaching the lowest level at 4 h after treatment (FIG. 20A). Concomitantly, Snail protein levels were significantly reduced at ≧4 h post-treatment with NPI-0052 (FIG. 20B). NPI-0052 interferes with Snail expression at both the mRNA and protein levels. The baseline RKIP and Snail levels in DU-145 cells were inversely correlated with Snail expression to be dominant over RKIP, according to the high metastatic potential of this cell line. Cell treatment with NPI-0052 maintained the inverse correlation between RKIP and Snail levels of expression.

The direct role of Snail repression by NPI-0052 in the inhibition of EMT was determined by using Snail siRNA transfected DU-145 cells (high basal levels of Snail). A random nucleotide sequence (CNTR siRNA) was used as a control in the transfection assays. Blots are representative of three independent and reproducible experiments. Transfection of DU-145 cells with Snail siRNA mimicked NPI-0052 with respect to morphological changes related to inhibition of the mesenchymal cell phenotype and acquisition of the epithelial-related morphology (FIG. 20C). The above changes were reflected by the induction of RKIP, E-cadherin and cytokeratin 18 protein levels and downregulation of vimentin and fibronectin gene expression, as assessed by western blot and IFA (FIGS. 20D, 20E). These findings corroborate earlier observations and suggest the role of Snail inhibition by NPI-0052 in the inhibition of EMT.

### Example 10

### Ectopic Expression of Snail in LNCaP Cells Represses RKIP Expression and Increases their Metastatic Potential

Overexpression of Snail in the nonmetastatic LNCaP cell line was examined to determine whether EMT would be induced and a more mesenchymal phenotype acquired, compared to wild type cells.

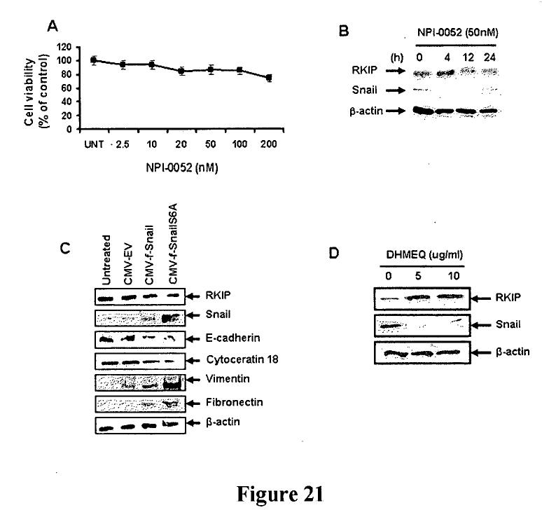

The effect of NPI-0052 on the levels of RKIP and Snail expression in LNCaP cells was determined. Subtoxic NPI-0052 concentrations were assessed using trypan blue exclusion assay (FIG. 21A). Compared to DU-145 cells, LNCaP cells exhibited higher RKIP and lower Snail expression. Treatment of LNCaP cells with 50 nM NPI-0052 resulted in an increase of RKIP protein levels at 4 h post-treatment, while the baseline low Snail level was completely undetectable at 4 and 12 h post-treatment and re-appeared at 24 h following treatment (FIG. 21B), namely, NPI-0052 treatment induced RKIP expression, concomitant with an inhibition of Snail expression. These results further suggest that the observed effect of NPI-0052 on the modification of RKIP and Snail expression could be dependent on the baseline levels of the above gene products in the cell line tested.

Since Snail is a highly unstable protein due to aberrant phosphorylation and proteasome degradation, Snail overexpression in LNCaP cells was performed using both the unstable wild type expression vector (CMV-f-Snail) and the stable vector carrying mutations in the phosphorylation sites (CMV-f-Snail S6A) (FIG. 21C). LNCaP cells were transfected with CMV-flag-Snail expression vectors (CMV-f-Snail) using lipofectamine, 24 h after transfection, cell lysates were harvested and subjected to immunoblot analysis. To circumvent the possible effect of the highly unstable Snail on RKIP expression or the expression of the other gene products tested, a mutated stable variant (Snail-6SA), which has all its six phosphorylable serine in the consensus GSK-3b sites mutated to alanine was used along with the wild-type Snail. A CMV-EV was used as control. From FIG. 21C, CMV-f-Snail-6SA induced the EMT phenotype in the non-metastatic LnCAP, however, ectopic Snail expression in LNCaP cells by CMV-f-Snail was less effective compared to CMV-f-Snail S6A in reducing the basal level of expression RKIP, E-cadherin and cytokeratin 18, and in inducing the level of expression of vimentin and fibronectin. These findings corroborated the above findings in DU-145 cells that NPI-0052 inhibition of Snail controls, at least in part, the suppressive effect on the EMT

### Example 11

### Direct Role of NF-κB Inhibition on the Regulation of Snail and RKIP Expression

The interrelationship among the NF-κB, Snail and RKIP gene products shown above to be modified by NPI-0052 and the inhibition of EMT was examined in DU-145 cells that were treated with the NF-κB inhibitor DHMEQ. DU-145 cells were treated with 5 or 10 μg/ml DHMEQ and protein lysates were harvested 24 h after treatment for RKIP and Snail protein determination by Western blot. Concomitant with the effect of NPI-0052 on the expression profiles of Snail and RKIP (FIGS. 19B and 19B), DHMEQ treatment resulted in Snail suppression and RKIP induction (FIG. 21D). These findings suggested the regulation by NPI-0052 of the NF-κB-Snail-RKIP loop and resulting inhibition of the EMT.

The examples described above are set forth solely to assist in the understanding of the embodiments. Thus, those skilled in the art will appreciate that the methods may provide derivatives of compounds.

One skilled in the art would readily appreciate that the present invention is well adapted to carry out the objects and obtain the ends and advantages mentioned, as well as those inherent therein. The methods and procedures described herein are presently representative of preferred embodiments and are exemplary and are not intended as limitations on the scope of the invention. Changes therein and other uses will occur to those skilled in the art which are encompassed within the spirit of the invention.

What is claimed is:

1. A method for inhibiting metastasis in a subject having a metastatic condition, comprising:
identifying a subject experiencing or at risk for metastasis; and
administering to the subject an effective amount of a compound of Formula I, or a pharmaceutically acceptable salt or pro-drug ester thereof, wherein the compound of Formula I has the structure:

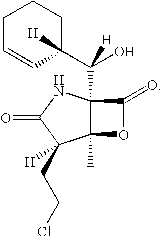

2. The method of claim 1, wherein said metastatic condition is selected from the group comprising prostate cancer, lung cancer, breast cancer, melanoma, colon cancer, kidney cancer, and pancreatic cancer.

3. The method of claim 1, wherein said metastatic condition comprises prostate cancer.

4. The method of claim 1, wherein said compound of Formula I is administered in combination with an effective amount of an additional anticancer agent.

5. The method of claim 4, wherein said additional anticancer agent is selected from the group comprising vincristine, cis-diamminedichloridoplatinum(II) (CDDP), Tumor necrosis (TNF)-related apoptosis-inducing ligand (TRAIL), and agonist antibody to DR4 or DR5.

6. The method of claim 1, wherein said subject is a mammal.

7. The method of claim 1, wherein said subject is human.

8. A method for evaluating the metastatic potential of a cancer comprising measuring the expression level of at least one marker in a sample from a subject afflicted with said cancer, wherein said at least one marker is selected from RKIP, Snail, NF-κB, E-cadherin, cytokeratin 18, fibronectin, and vimentin.

9. The method of claim 8, wherein said measuring the expression level of at least one marker comprises measuring the expression level of a nucleic acid.

10. The method of claim 8, wherein said measuring the expression level of at least one marker comprises measuring the expression level of a protein.

11. The method of claim 8, further comprising comparing said expression level of said at least one marker in said sample to the expression level of said at least one marker in normal tissue, tissue from a known stage of cancer, or cancerous tissue with a known metastatic potential.

12. The method of claim 11, wherein said at least one marker comprises RKIP.

13. The method of claim 12, wherein said at least one marker comprises Snail.

14. The method of claim 12, wherein a decrease in the expression level of said RKIP indicates the metastatic potential of said cancer.

15. The method of claim 11, wherein said at least one marker comprises at least three markers.

16. The method of claim 11, wherein said at least one marker comprises at least four markers.

17. The method of claim 11, wherein said at least one marker comprises at least five markers.

18. The method of claim 11, wherein said at least one marker comprises at least six markers.

19. The method of claim 11, further comprising administering to the subject an effective amount of a compound of Formula I, or a pharmaceutically acceptable salt or pro-drug ester thereof, wherein the compound of Formula I has the structure: [chemical structure]
